(19) United States
(12) Patent Application Publication
(10) Pub. No.: US 2016/0235813 A1
(43) Pub. Date: Aug. 18, 2016

(54) ANTI-INFLAMMATORY PROTEINS AND METHODS OF USE

(71) Applicant: JAMES COOK UNIVERSITY, Townsville, Queensland (AU)

(72) Inventors: Cinzia Cantacessi, Townsville (AU); Alex Loukas, Townsville (AU)

(21) Appl. No.: 15/023,108

(22) PCT Filed: Sep. 18, 2014
(86) PCT No.: PCT/AU2014/050238

§ 371 Date: Mar. 18, 2016

(30) Foreign Application Priority Data
Sep. 18, 2013 (AU) 2013903584

(51) Int. Cl. A61K 38/17; A61K 45/06

(52) U.S. Cl. CPC A61K 38/1767; A61K 45/06

(57) ABSTRACT

A method and composition for reducing or alleviating inflammation in a subject is provided, including administering to the subject a therapeutically effective amount; of one or more isolated proteins respectively comprising an amino acid sequence set forth in SEQ ID NOS:1-31, or a biologically active fragment or variant thereof or combinations of these to thereby reduce or alleviate inflammation. Inflammation may be associated with a disease is a disease of the digestive tract such as chronic gastritis or an inflammatory bowel disease such as Crohn's disease or ulcerative colitis, or a disease of the respiratory system, such as asthma, emphysema, chronic bronchitis, and chronic obstructive pulmonary disease.

### TECHNICAL FIELD

THIS INVENTION relates to isolated proteins for preventing and/or treating inflammation. More particularly, this invention relates to the use of tissue metalloprotease inhibitor proteins for reducing, alleviating and/or preventing inflammation.

### BACKGROUND

Inflammation is a non-specific reaction mounted by the immune system in response to a perceived injury or threat. It is an innate defensive response, distinguished from the more precisely tailored adaptive responses of the immune system. Inflammation may work cooperatively with adaptive responses of the immune system, which develop more slowly but are more precisely targeted to a harmful agent such as a pathogen that may be causing localised injury.

While associated with infection, inflammation occurs in response to many types of injury, including physical trauma, burns (e.g., from radiation, heat or corrosive materials), chemical or particulate irritants, bacterial or viral pathogens, and localized oxygen deprivation (ischemia) inflammation is also associated with autoimmune diseases and allergic reactions. Inflammation includes the classic symptoms of redness, heat, swelling, and pain, and may be accompanied by decreased function of the inflamed organ or tissue.

While a number of methods for treating inflammation are known, all of them have limitations, particularly with regard to broad based efficacy. Thus, there is a need for new methods for reducing, alleviating and/or preventing inflammation associated with a variety of causes.

### SUMMARY

The present invention is directed to methods and compositions for treating and/or preventing inflammation and/or diseases or conditions associated with inflammation.

In a broad form, the invention relates to use of one or more tissue metalloprotease inhibitor (TMP) proteins, for reducing, alleviating and/or preventing inflammation and/or diseases or conditions associated with inflammation such as asthma and/or inflammatory bowel disease.

In one aspect, the invention provides a method of reducing or alleviating inflammation in a subject, the method including the step of administering to the subject a therapeutically effective amount of an isolated protein comprising an amino acid sequence set forth in FIG. 1 and/or FIG. 2, a biologically active fragment, variant or derivative thereof or a combination of these, to thereby reduce or alleviate inflammation in the subject

Preferably, the isolated protein comprises an amino acid sequence set forth in any one of SEQ ID NOS:1-31.

In one embodiment, this aspect further includes the step of administering to the subject at least one additional agent.

Suitably, according to the above embodiment, the at least one additional agent is selected from the group consisting of nonsteroidal anti-inflammatory drugs (NSAIDs), aminosalicylates, corticosteroids, immunosuppressants, anti-cytokine/cytokine receptor agents (e.g., anti-TNFα agents, anti-IL-5 agents, anti-IL-13 agents, anti-IL-17 agents, and anti-IL-6R agents), antibiotics, and combinations thereof.

In some embodiments, the inflammation is associated with or secondary to a disease, disorder and/or condition in the subject, particularly an immunological disease, disorder and/or condition.

In certain embodiments the disease is a disease of the digestive tract or the respiratory system.

In another embodiment, the disease, disorder and/or condition is refractory to a baseline therapy.

Suitably, according to the above embodiment, the baseline therapy comprises administration of at least one baseline agent selected from the group consisting of nonsteroidal anti-inflammatory drugs (NSAIDs), aminosalicylates, corticosteroids, immunosuppressants, anti-cytokine/cytokine receptor agents (e.g., anti-TNFα agents, anti-IL-5 agents, anti-IL-13 agents, anti-IL-17 agents, and anti-IL-6R agents), antibiotics, and combinations thereof.

In another aspect, the invention provides a method of preventing inflammation in a subject, the method including the step of administering to the subject a therapeutically effective amount of an isolated protein comprising an amino acid sequence set forth in any one of SEQ ID NOS:1-31, a biologically active fragment or variant thereof, or a combination of these, to thereby reduce or alleviate inflammation in the subject

In one embodiment, this aspect further includes the step of administering to the subject at least one additional agent.

Preferably, the subject is a mammal.

More preferably, the subject is a human.

A further aspect of the invention provides a pharmaceutical composition comprising a therapeutically effective amount of an isolated protein comprising an amino acid sequence set forth in FIG. 1 and/or FIG. 2, a biologically active fragment, variant or derivative thereof, or a combination of these, together with a pharmaceutically acceptable carrier, diluent or excipient.

Preferably, the isolated protein comprises an amino acid sequence set forth in any one of SEQ ID NOS:1-31.

In some embodiments, the pharmaceutical composition may further comprise at least one additional agent.

The at least one additional agent may be selected from the group consisting of nonsteroidal anti-inflammatory drugs (NSAIDs), aminosalicylates, corticosteroids, immunosuppressants, anti-cytokine/cytokine receptor agents (e.g., anti-TNFα agents, anti-IL-5 agents, anti-IL-13 agents, anti-IL-17 agents, and anti-IL-6R agents), antibiotics, and combinations thereof.

Suitably, the pharmaceutical composition is for preventing or treating inflammation and/or for preventing or treating a disease or condition associated with inflammation.

Related aspects of the invention include an isolated protein comprising a biologically active fragment of an amino acid sequence set forth in FIGS. 1 and 2, such as SEQ ID NOS:1-31; an isolated nucleic acid encoding the isolated protein; a genetic construct comprising the isolated nucleic acid; and/or a host cell comprising the genetic construct.

Throughout this specification, unless the context requires otherwise, the words “comprise”, “comprises” and “comprising” will be understood to imply the inclusion of a stated integer or group of integers but not the exclusion of any other integer or group of integers.

As used in this specification the indefinite articles “a” and “an” may refer to one entity or a plurality of entities (e.g. proteins) and are not to be read or understood as being limited to a single entity.

### BRIEF DESCRIPTION OF THE FIGURES

FIG. 1. Amino acid sequences designated SEQ ID NOS: 1-33.

FIG. 2: Amino acid sequence alignment of tissue inhibitors of metalloproteases (TIMPs) based on predictions of their secondary structures. Homo sapiens TIMP-1 (GenBank accession number XP_010392.1), TIMP-2 (NP_003246.1), TIMP-3 (P35625.2), TIMP-4 (Q99727.1), Canis familiaris TIMP-2 (AF112115.1), Gallus gallus TIMP-2 (AAB69168.1), Oryctolagus cuniculus TIMP-2 (AAB35920.1), Mus musculus TIMP-1 (P12032.2), TIMP-2 (P25785.2), TIMP-3 (P39876.1), TIMP-4 (Q9JHB3.1), Drosophila melanogaster TIMP (AAL39356.1), Caenorhabditis elegans CRI-2 (K07C11.5), Ancylostoma caninum TMP-1 (AF372651.1), TMP-2 (EU523696.1), Ancylostoma duodenale TIMP-1 (ABP88131.1), Necator americanus (NECAME_13168, NECAME_07191, NECAME_01063, NECAME_05356, NECAME_05357, NECAME_14664, NECAME_08457 and NECAME_08458), Dictyocaulus filaria (1495356.2; http://www.gasserlab.org), Oesophagostomum dentatum (E59TEJM01BU99S and E59TEJM02GRTKW; http://www.gasserlab.org), Ascaris suum (GS_21732, GS_04796, GS_08199; http://www.wormbase.org), Schistosoma haematobium A_01727, Schistosoma mansoni Smp_087690 and Schistosoma japonicum Sjp_0053050 (http://www.genedb.org). Ancylostoma ceylanicum AceES-2 (GenBank Q6R7N7) is also included.

FIG. 3: Structural comparison of four netrin domain-containing proteins. The netrin domains of Ac-TMP-2 (homology model based on Hs-TIMP-2), Hs-TIMP-2 (PDB accession code 1br9), AceES-2 (PDB accession code 3nsw) and Sh-TIMP (A_01727; homology model based on Hs-TIMP-2) are coloured blue, cysteine side chain residues are, rendered as yellow sticks. Red highlighted areas indicate regions of interactions with MMPs; these regions are inferred for Ac-TMP-2, AceES-2 and Sh-TIMP based on the alignment in FIG. 2. The parasite proteins Ac-TMP-2 and Sh-TIMP and human Hs-TIMP-2 share the same intra-domain disulphide bonding pattern. In contrast, AceES-2 possesses a different pattern with two intra-molecular disulphide bonds. The disulphide bond engaging the N-terminal cysteine (Cys3-Cys62) is reminiscent of that found in Ac-TMP-2, Sh-TIMP and Hs-TIMP-2. The other disulphide bond (Cys77-Cys84) is unique to AceES-2. The C-terminal domain of Hs-TIMP-2 is rendered magenta. The C-terminal domains of Ac-TMP-2 and Sh-TIMP are shown in grey for illustration only and the three-dimensional structures of these domains are neither based on computational, nor experimental evidence. Comparative modeling was performed using MODELLER [59] based on the structure-based sequence alignment shown in FIG. 2.

FIG. 4: The phylogenetic relationships of tissue inhibitor of metalloproteases (TIMPs) based on Bayesian Inference. The posterior probability supporting each clade is indicated. Homo sapiens TIMP-1 (GenBank accession number XP_010392.1), TIMP-2 (NP_003246.1), TIMP-3 (P35625.2), TIMP-4 (Q99727.1), Gallus gallus TIMP-2 (AAB69168.1), Canis familiaris TIMP-2 (AF112115.1), Oryctolagus cuniculus TIMP-2 (AAB35920.1), Drosophila melanogaster TIMP (AAL39356.1), Mus musculus TIMP-1 (P12032.2), TIMP-2 (P25785.2), TIMP-3 (P39876.1), TIMP-4 (Q9JHB3.1), Caenorhabditis elegans CRI-2 (K07C11.5), Ancylostoma caninum TMP-1 (AF372651.1), TMP-2 (EU523696.1), Ancylostoma duodenale TIMP-1 (ABP88131.1), Necator americanus (NECAME_13168, NECAME_07191, NECAME_01063, NECAME_05356, NECAME_05357, NECAME_14664, NECAME_08457 and NECAME_08458), Dictyocaulus filaria (1495356.2; http://www.gasserlab.org), Oesophagostomum dentatum (E59TEJM01BU99S and E59TEJM02GRTKW; http://www.gasserlab.org) and Ascaris suum (GS_21732, GS_021796, GS_08199; http://www.wormbase.org).

### DETAILED DESCRIPTION

The present invention relates to methods for reducing, alleviating and/or preventing inflammation and/or inflammatory diseases or conditions such as asthma and/or inflammatory bowel disease.

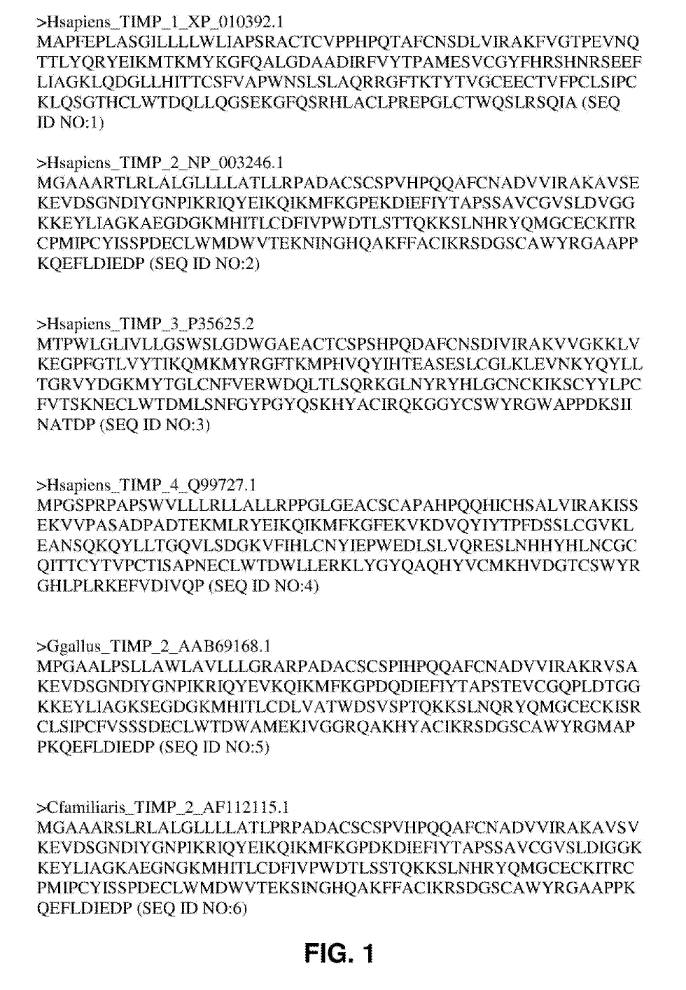

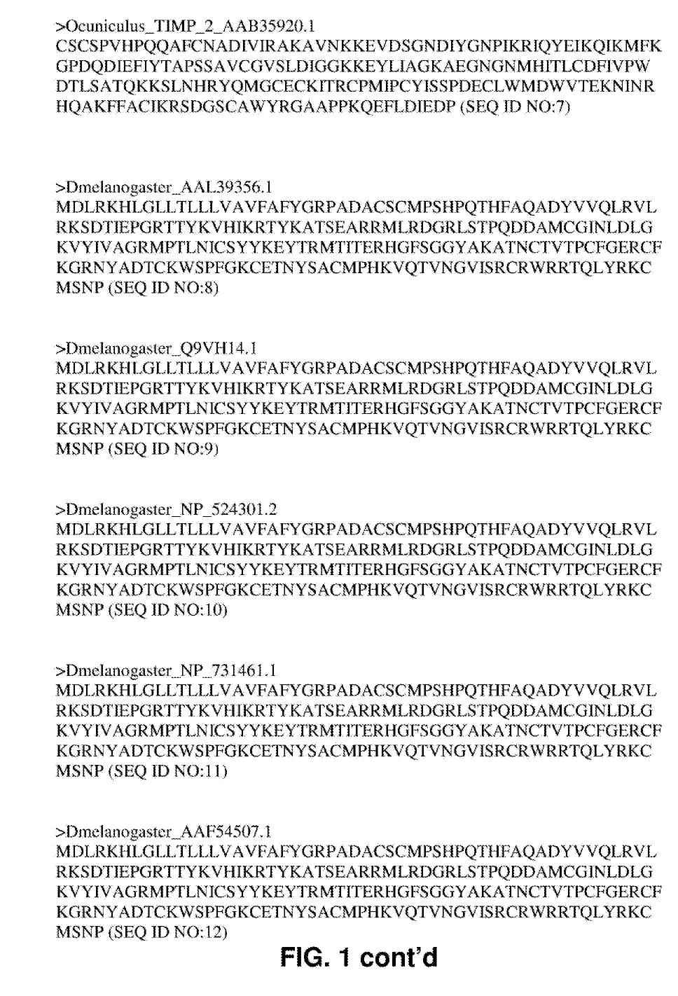

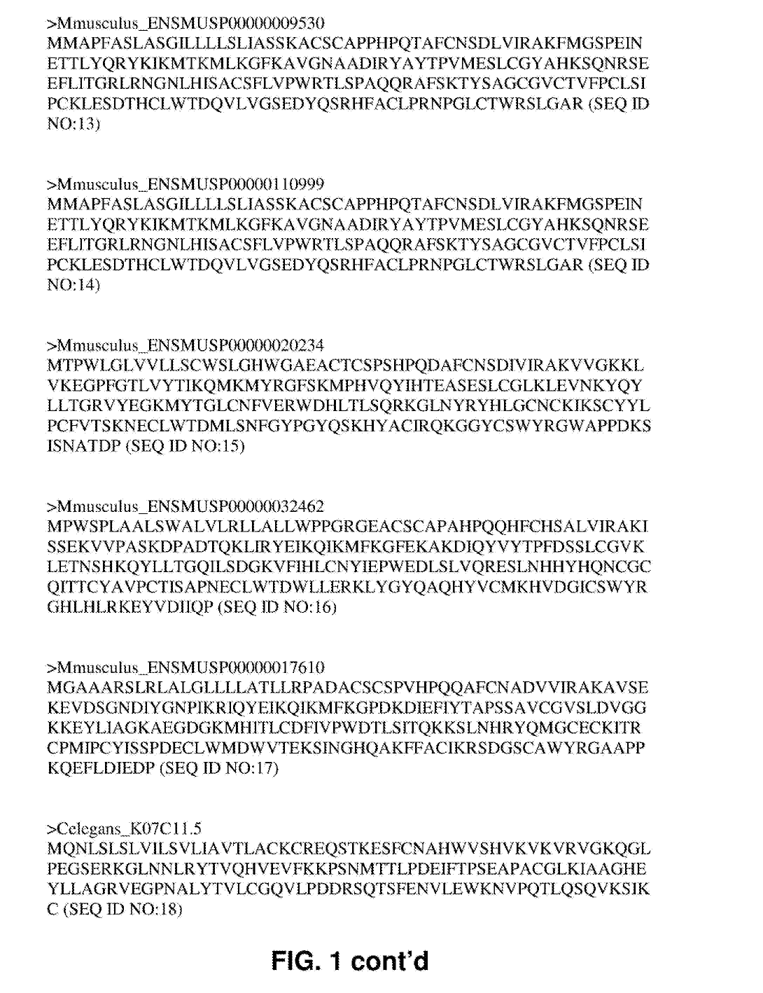

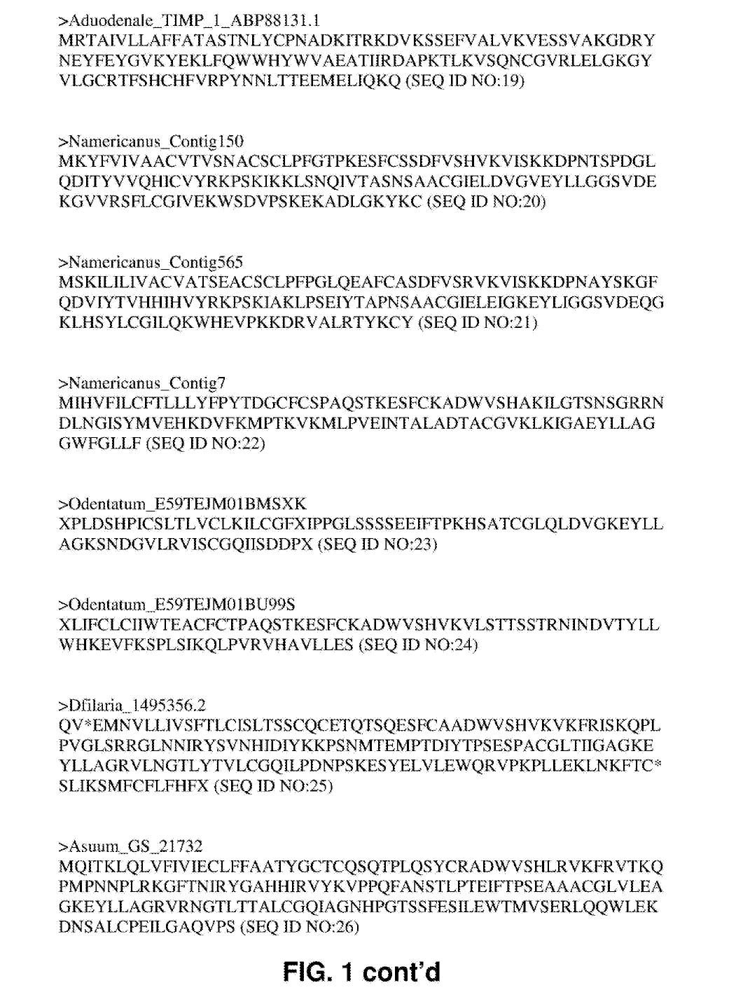

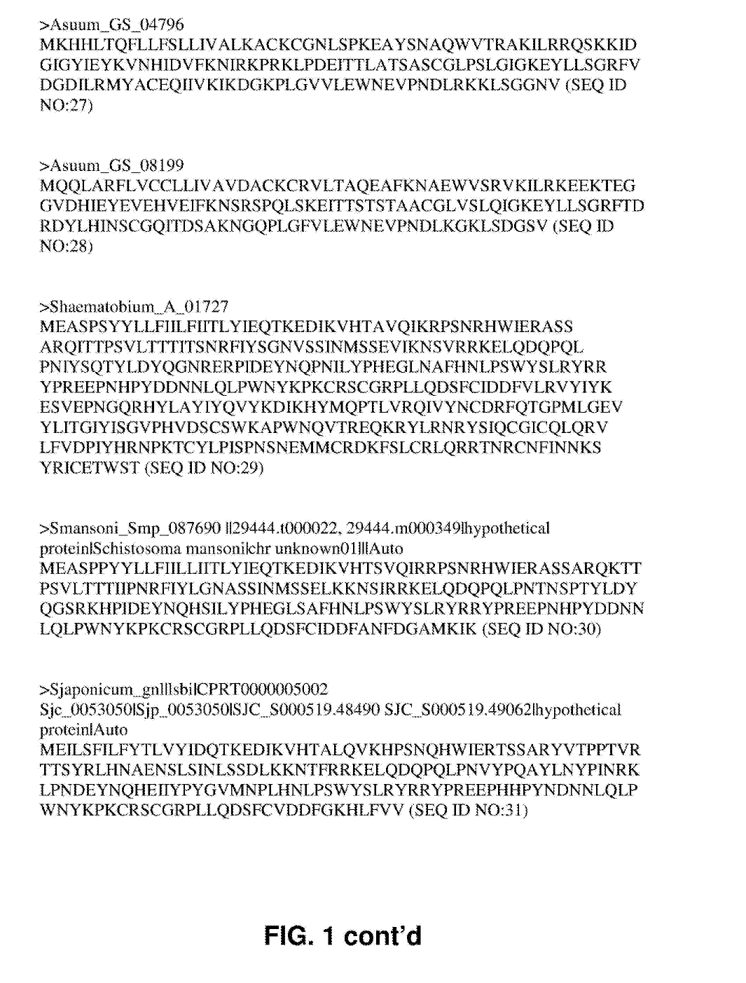

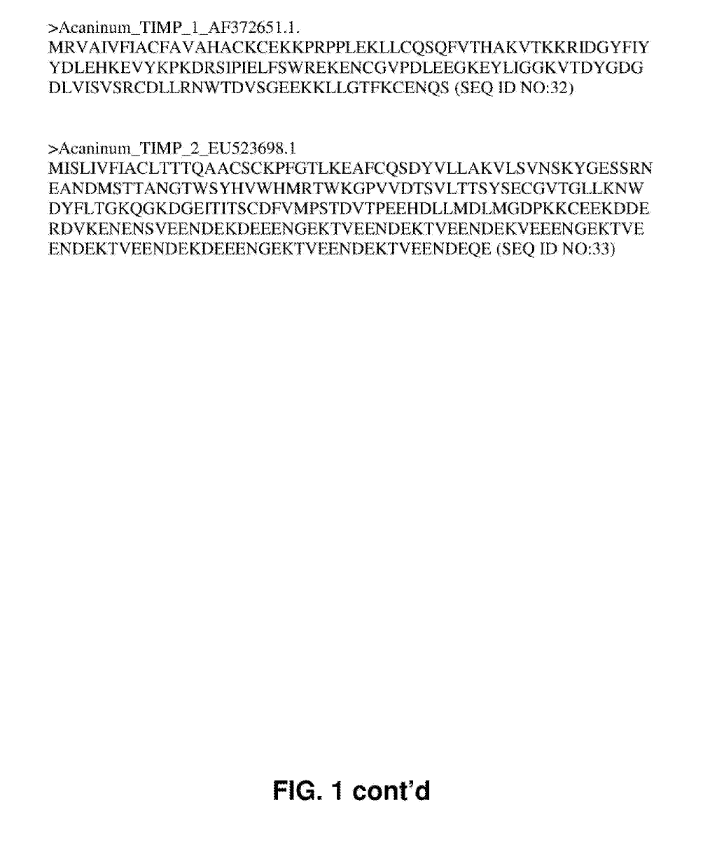

The invention is at least partly predicated on the unexpected discovery that one or more tissue metalloprotease inhibitor proteins (TMP) comprising the amino acid sequences set forth in FIGS. 1 and 2, such as SEQ ID NOS:1-31, may be useful for reducing, alleviating and/or preventing inflammation and/or inflammatory diseases or conditions in a subject.

The proteins of FIGS. 1 and 2, such as SEQ ID NOS:1-31, are obtainable from any of a plurality of different animal phyla, classes, orders, genera and/or species inclusive of mammals such as humans, dogs and mice, avians such as chickens, insects, worms and protozoa.

In particular aspects, the invention contemplates use of one or more isolated proteins respectively comprising an amino acid sequence set forth in FIGS. 1 and/or 2, such as SEQ ID NOS:1-31, or a biologically active fragment or variant thereof or combinations of these for reducing, alleviating and/or preventing inflammation and/or inflammatory disease or conditions.

While the isolated proteins comprising respective amino acid sequences set forth in FIGS. 1 and 2, such as SEQ ID NOS:1-31, may be collectively referred to as tissue inhibitors of metalloproteases or “TMP” or “TIMP” proteins, it should be understood that the one or more isolated proteins do not necessarily possess this particular biological activity. Furthermore, even if the one or more proteins have this biological activity, it is not necessarily essential or required for the anti-inflammatory properties of the isolated proteins.

In one aspect, the invention provides a method of reducing or alleviating inflammation in a subject, the method including the step of administering to the subject a therapeutically effective amount of one or more isolated proteins respectively comprising an amino acid sequence set forth in FIGS. 1 and/or 2, such as SEQ ID NOS:1-31, or a biologically active fragment, derivative or variant thereof or combinations of these to thereby reduce or alleviate inflammation in the subject.

In another aspect, the invention provides a method of preventing inflammation in a subject, the method including the step of administering to the subject a therapeutically effective amount of one or more isolated proteins respectively comprising an amino acid sequence set forth in FIGS. 1 and/or 2, such as SEQ ID NOS:1-31, or a biologically active fragment, derivative or variant thereof or combinations of these to thereby prevent inflammation in the subject.

By “reducing”, as in reducing inflammation in a subject, is meant a lessening or shortening of a symptom, aspect, or characteristic associated with inflammation (e.g., redness, heat, swelling, and/or pain), or of the length of time a subject experiences a symptom, aspect, or characteristic associated with inflammation. Such reducing need not be absolute to be beneficial to the subject. By “alleviating”, as in alleviating inflammation in a subject, is meant a reduction in the severity or seriousness of a symptom, aspect, or characteristic associated with inflammation (e.g., redness, heat, swelling, and/or pain). Such alleviating need not be absolute to be beneficial to the subject. Reduction and/or alleviation of inflammation in a subject can be determined using any methods or standards known to the ordinarily skilled artisan, including both qualitative and quantitative methods and standards.

It is to be understood that reducing or alleviating inflammation in a subject is a method of treating inflammation in the subject. As used herein, “treating” (or “treat” or “treatment”) refers to a therapeutic intervention that ameliorates a sign or symptom of inflammation after it has begun to develop. The term “ameliorating,” with reference to inflammation, refers to any observable beneficial effect of the treatment. The beneficial effect can be determined using any methods or standards known to the ordinarily skilled artisan.

As used herein, “preventing” (or “prevent” or “prevention”) refers to a course of action initiated prior to the onset of a symptom, aspect, or characteristic of inflammation so as to prevent or reduce the symptom, aspect, or characteristic. It is to be understood that such preventing need not be absolute to be beneficial to a subject. A “prophylactic” treatment is a treatment administered to a subject who does not exhibit signs of inflammation or exhibits only early signs for the purpose of decreasing the risk of developing a symptom, aspect, or characteristic of inflammation.

As used herein, “inflammation” refers to the well known localised response to various types of injury or infection, which is characterised by redness, heat, swelling, and pain, and often also including dysfunction or reduced mobility. Inflammation represents an early defence mechanism to contain an infection and prevent its spread from the initial focus. Major events in inflammation include dilation of capillaries to increase blood flow, changes in the microvasculatum structure, leading to escape of plasma and proteins and leukocytes from the circulation, and leukocyte emigration from the capillaries arid accumulation at the site of injury or infection.

Inflammation is often associated with, or secondary to, a disease, disorder and/or condition in a subject, including an immunological disease, disorder and/or condition (such as an autoimmune disease, disorder and/or condition) and allergic reactions. Exemplary immunological diseases, disorders and/or conditions include, without limitation, Addison's disease, ankylosing spondylitis, celiac disease, chronic inflammatory demyelinating polyneuropathy (CIDP), chronic recurrent multifocal ostomyelitis (CRMO), Crohn's disease, demyelinating neuropathies, glomerulonephritis, Goodpasture's syndrome, Graves' disease, Guillain-Barre syndrome, Hashimoto's encephalitis, Hashimoto's thyroiditis, hypogammaglobulinemia, idiopathic thrombocytopenic purpura (ITP), insulin-dependent diabetes (type 1), juvenile arthritis, Kawasaki syndrome, multiple sclerosis, myasthenia gravis, postmyocardial infarction syndrome, primary biliary cirrhosis, psoriasis, idiopathic pulmonary fibrosis, Reiter's syndrome, rheumatoid arthritis, sarcoidosis, scleroderma, Sjogren's syndrome, systemic lupus erythematosus (SLE), thrombocytopenic purpura (TTP), ulcerative colitis, vasculitis, vitiligo, and Wegener's granulomatosis.

As will be understood by one of ordinary skill in the art, diseases of the digestive tract (e.g., chronic gastritis or an inflammatory bowel disease, such as, Crohn's disease or ulcerative colitis) and diseases of the respiratory system (e.g., asthma, emphysema, chronic bronchitis, and chronic obstructive pulmonary disease (COPD)) have an inflammatory component, and thus are particularly amenable to treatment using the disclosed methods.

In one embodiment, the invention provides a method of treating and/or preventing an inflammatory bowel disease in a subject. In one embodiment, the inflammatory bowel disease is Crohn's disease or ulcerative colitis.

In another embodiment, the invention provides a method of treating and/or preventing asthma in a subject.

As will also be understood by one of ordinary skill in the art, inflammation that is associated with, or secondary to, a disease, disorder and/or condition in a subject, often occurs when the disease, disorder and/or condition is refractory to a baseline therapy, for example, a baseline therapy comprising nonsteroidal anti-inflammatory drugs (NSAIDs), aminosalicylates, corticosteroids, immunosuppressants anti-cytokine/cytokine receptor agents (e.g., anti-TNFα agents, anti-IL-5 agents, anti-IL-13 agents, anti-IL-17 agents, and anti-IL-6R agents), antibiotics, and combinations thereof. By “refractory” is intended resistance to treatment, particularly first line treatment.

The term “subject” includes both human and veterinary subjects. For example, administration to a subject can include administration to a human subject, or a veterinary subject. Preferably, the subject is a human. However, therapeutic uses according to the invention may also be applicable to mammals such as domestic and companion animals, performance animals such as horses, livestock, and laboratory animals.

By “administration” is intended the introduction of a composition (e.g., a pharmaceutical composition comprising one or more isolated proteins respectively comprising an amino acid sequence set forth in FIGS. 1 and/or 2, such as SEQ ID NOS:1-31, or a biologically active fragment, derivative or variant thereof or combinations of these to thereby reduce or alleviate inflammation in the subject) into a subject by a chosen route.

The term “therapeutically effective amount” describes a quantity of a specified agent sufficient to achieve a desired effect in a subject being treated with that agent. For example, this can be the amount of a composition comprising one or more isolated proteins respectively comprising an amino acid sequence set forth in FIGS. 1 and/or 2, such as SEQ ID NOS:1-31, or a biologically active fragment, derivative or variant thereof or combinations of these, necessary to reduce, alleviate and/or prevent inflammation. In some embodiments, a “therapeutically effective amount” is sufficient to reduce or eliminate a symptom of inflammation. In other embodiments, a “therapeutically effective amount” is an amount sufficient to achieve, a desired biological effect, for example an amount that is effective to decrease redness, heat, swelling, and/or pain associated with inflammation.

Ideally, a therapeutically effective amount of an agent is an amount sufficient to induce the desired result without causing a substantial cytotoxic effect in the subject. The effective amount of an agent, for example one or more isolated proteins respectively comprising an amino acid sequence set forth in FIGS. 1 and/or 2, such as SEQ ID NOS:1-31, or a biologically active fragment or variant thereof or combinations of these, useful for reducing, alleviating and/or preventing inflammation will be dependent on the subject being treated, the type and severity of any associated disease, disorder and/or condition, and the manner of administration of the therapeutic composition.

A therapeutically effective amount of a composition comprising one or more isolated proteins respectively comprising an amino acid sequence set forth in FIGS. 1 and/or 2, such as SEQ ID NOS:1-31, or a biologically active fragment or variant thereof or combinations of these may be administered in a single dose, or in several doses, for example daily, during a course of treatment. However, the frequency of administration is dependent on the preparation applied, the subject being treated, the severity of inflammation, and the manner of administration of the therapy or composition.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material includes material in native and recombinant form. The term “isolated” also encompasses terms such as “enriched”, “purified” and/or “synthetic”. Synthetic includes recombinant synthetic and chemical synthetic.

As used herein, “fragment” describes a domain, portion, region or sub-sequence of an isolated protein comprising no more than 6, 10, 12, 15, 20, 30, 40, 50 60, 70, 80, 90, 100, 110, 120, 130, 140, 150, 160, 170, 180 or 190 contiguous amino acids of any one of the proteins set forth in FIGS. 1 and 2, such as SEQ ID NOS:1-31.

In one particular embodiment, the fragment is, or corresponds to, an N-terminal domain, portion, sub-sequence or region of an isolated protein comprising an amino acid sequence set forth in FIGS. 1 and/or 2, such as SEQ ID NOS:1-31. Suitably, one or a plurality of N and/or C-terminal amino acids may be deleted without substantially diminishing anti-inflammatory activity. For example, the truncated polypeptide or protein may lack at least 5, 10, 15, 20, 25, 30, 35, 40, 45, 50, 55, 60, 65, 70, 75, 80, 85, 90, 95 or 100 or more N and/or C-terminal amino acids, that are normally present in the full length or wild-type protein or polypeptide.

In one embodiment, one or more N-terminal amino acids may be deleted or absent. In some embodiments, the N terminal amino acids are of a signal peptide which may be deleted or replaced with a heterologous signal peptide amino acid sequence (e.g such as for yeast expression). For example, the truncated polypeptide or protein may lack at least 1, 2, 3, 4, 5, 6, 7, 8, 9, 10, 11, 12, 13, 14, 15, 16, 17, 18, 19, 20 or more N-terminal amino acids normally present in the TMP protein.

Suitably, the truncated polypeptide or protein comprises the amino acid sequence C-X-C at or near the N-terminus. In this regard “near the N-terminus” means N-terminal or within about 1, 2, 3, 4, 5, 6, 7, 8, 9 or 10 amino acids of the N-terminus.

While not wishing to be bound by any particular theory, it is proposed that C-terminal amino acids may be deleted, particularly from a wild-type TMP2, alone or together with some N-terminal amino acids, as long as the C-X-C motif at or near the N-terminus is retained to allow insertion into the MMP active site cleft with subsequent inhibition of catalytic activity.

While the proteins of FIGS. 1 and 2, such as SEQ ID NOS:1-31, may be referred to as tissue inhibitors of metalloproteases, it should be understood that such proteins do not not necessarily possess this particular biological activity. Furthermore, even any or all of the proteins have this biological activity, it is not necessarily essential or required for the anti-inflammatory properties of the protein.

Preferably, the fragment is a “biologically active fragment”. In some embodiments, the biologically active fragment has no less than 10%, preferably no less than 25%, more preferably no less than 50%, and even more preferably no less than 75%, 80%, 85%, 90%, or 95% of the anti-inflammatory activity of the isolated protein. Such activity may be evaluated using standard testing methods and bioassays recognizable by the skilled artisan in the field as generally being useful for identifying such activity.

In some embodiments, an isolated protein may comprise a plurality of the same or different fragments, inclusive of biologically active fragments.

Also contemplated are variants of any one of the isolated proteins comprising an amino acid sequence set forth in FIGS. 1 and/or 2, such as SEQ ID NOS:1-31.

Typically, and in relation to proteins, a “variant” protein has one or more amino acids that have been replaced by different amino acids. It is well understood in the art that some amino acids may be changed to others with broadly similar properties without changing the nature of the activity of the protein (i.e., conservative substitutions).

It will also be appreciated that one or more amino acid residues of a may be modified or deleted, or additional sequences added, without substantially altering the functional and/or biological activity of the isolated protein or fragment thereof. Such activity may be evaluated using standard testing methods and bioassays recognizable by the skilled artisan in the field as generally being useful for identifying such activity.

The term “variant” includes peptidomimetics and orthologs of an isolated protein comprising an amino acid sequence set forth in SEQ ID NOS:1-31. By “peptidomimetic” is meant a molecule containing non-peptidic structural elements that are capable of mimicking or antagonising the biological action(s) of a natural parent peptide. Examples of peptidomimetics include peptidic compounds in which the peptide backbone is substituted with one or more benzodiazepine molecules (see, e.g., James et al., Science 260:1937-42, 1993) and “retro-inverso” peptides (see, e.g., U.S. Pat. No. 4,522,752). The term also refers to a moiety, other than a naturally occurring amino acid, that conformationally and functionally serves as a substitute for a particular amino acid in a protein without adversely interfering to a significant extent with the function of the protein. Examples of amino acid mimetics include D-amino acids. Proteins substituted with one or more D-amino acids may be made using well known peptide synthesis procedures. Additional substitutions include amino acid analogs having variant side chains with functional groups, such as, for example, b-cyanoalanine, canavanine, djenkolic acid, norleucine, 3-phosphoserine, homoserine, dihydroxyphenylalanine, 5-hydroxytryptophan, 1-methylhistidine, and 3-methylhistidine.

By “orthologs” of is meant structurally related proteins from the same or different organisms from which the proteins of FIGS. 1 and 2, such as SEQ ID NOS:1-31, were obtained or derived.

In one embodiment, a protein variant or ortholog shares at least 70%, preferably at least 75%, 80% or 85% and more preferably at least 90%, 91%, 92%, 93%, 94%, 95%, 96%, 97%, 98%, or 99% sequence identity with an amino acid sequence set forth in FIGS. 1 and 2, such as SEQ ID NOS:1-31.

Preferably, sequence identity is measured over at least 60%, more preferably over at least 75%, more preferably over at least 90% or more preferably over at least 95%, 98% or substantially the full length of a reference sequence consisting of an amino acid sequence set forth in SEQ ID NOS:1-31.

In order to determine percent sequence identity, optimal alignment of amino acid and/or nucleotide sequences may be conducted by computerised implementations of algorithms (Gcneworks program by Intelligcnetics; GAP, BESTFIT, FASTA, and TFASTA in the Wisconsin Genetics Software Package Release 7.0, Genetics Computer Group, WI, USA) or by inspection and the best alignment (i.e., resulting in the highest percentage homology over the comparison window) generated by any of the various methods selected. Reference also may be made to the BLAST family of programs as for example disclosed by Altschul et al., Nucl. Acids Res. 25:3389-402, 1997.

A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999).

A non-limiting example of a particular variant contemplated by the present invention is a non-glycosylated variant wherein an amino acid that is a site of glycosylation is deleted or replaced with another amino acid. Referring to SEQ ID NO:33, the amino acid sequence MSTTANGTWSYH (SEQ ID NO:35) comprises the bolded N-linked glycosylation site which may be mutated to a non-glycosylated amino acid, such as to a glutamine (Gln or Q) residue. Similar mutations may be incorporated into one or more of SEQ ID NOS:1-31.

Variant proteins can be produced by a variety of standard, mutagenic procedures known to one of skill in the art. A mutation can involve the modification of the nucleotide sequence of a single gene, blocks of genes or a whole chromosome, with the subsequent production of one or more mutant proteins. Changes in single genes may be the consequence of point mutations, which involve the removal, addition or substitution of a single nucleotide base within a DNA sequence, or they may be the consequence of changes involving the insertion or deletion of large numbers of nucleotides.

Mutations occur following exposure to chemical or physical mutagens. Such mutation-inducing agents include ionizing radiation, ultraviolet light and a diverse array of chemical agents, such as alkylating agents and polycyclic aromatic hydrocarbons, all of which are capable of interacting either directly or indirectly (generally following some metabolic biotransformations) with nucleic acids. The DNA lesions induced by such environmental agents may lead to modifications of base sequence when the affected DNA is replicated or repaired and thus to a mutation, which can subsequently be reflected at the protein level. Mutation also can be site-directed through the use of particular targeting methods.

Mutagenic procedures of use in producing isolated proteins comprising one or more mutations include, but are not limited to, random mutagenesis (e.g., insertional mutagenesis based on the inactivation of a gene via insertion of a known DNA fragment, chemical mutagenesis, radiation mutagenesis, error prone PCR (Cadwell and Joyce, PCR Methods Appl. 2:28-33, 1992)) and site-directed mutagenesis (e.g., using specific oligonucleotide primer sequences that encode the DNA sequence of the desired mutation). Additional methods of site-directed mutagenesis are disclosed in U.S. Pat. Nos. 5,220,007; 5,284,760; 5,354,670; 5,366,878; 5,389,514; 5,635,377; and 5,789,166.

Also provided are “derivatives” of the isolated proteins, biologically active fragments and variants. Such derivatives may include chemically modified proteins (e.g amino acid side chain modifications), chemically cross-linked proteins, proteins modified to include avidin, biotin and other binding moieties, addition of epitope tags and/or fusion partners (e.g FLAG, haemagglutinin, myc tags, GST or MBP, hexahistidine fusion partners), labels (e.g. radioactive labels, fluorescent labels) and enzymes (e.g HRP, alkaline phosphatase), although without limitation thereto.

Isolated proteins (inclusive of fragments, variants and derivatives) can be prepared by any suitable procedure known to those of skill in the art.

In one embodiment, isolated proteins (inclusive of fragments, variants and derivatives) are produced by chemical synthesis. Chemical synthesis techniques are well known in the art, although the skilled person may refer to Chapter 18 of CURRENT PROTOCOLS IN PROTEIN SCIENCE Eds. Coligan et. al., John Wiley & Sons NY (1995-2001) for examples of suitable methodology.

In another embodiment, the isolated proteins (inclusive of fragments, variants and derivatives) are prepared as recombinant proteins.

Another aspect of the invention therefore relates to an isolated nucleic acid encoding the isolated protein or a fragment thereof.

As used herein a “nucleic acid” may be single- or double-stranded DNA inclusive of cDNA and genomic DNA or RNA inclusive of mRNA. Suitably, for expression of the nucleic acid (such as for recombinant protein expression), a genetic construct may comprise the isolated nucleic acid operably linked or connected to one or more other nucleotide sequences. Such nucleotide sequences may include regulatory nucleotide sequences such as promoters, enhancers, polyadenylation sequences, splice sites, translation initiation or termination sequences, antibiotic resistances genes and selection marker genes although without limitation thereto. Promoters are typically selected according to a host cell used for expression, such as yeast, bacterial, insect, plant or mammalian host cells. Fusion partner or epitope tage sequences may also be added, such as hexahistidine, MBP, GST, haemagglutinin, FLAG and/or c-myc sequences. The genetic construct is suitably manipulated, propagated and/or expressed in a host cell engineered or manipulated to comprise the genetic construct. Such host cells may include yeast, bacterial, insect, plant or mammalian host cells, although without limitation thereto.

While production of recombinant proteins is well known in the art, the skilled person may refer to standard protocols as for example described in Sambrook et al., MOLECULAR CLONING. A Laboratory Manual (Cold Spring Harbor Press, 1989), in particular Sections 16 and 17; CURRENT PROTOCOLS IN MOLECULAR BIOLOGY Eds. Ausubel et al., (John Wiley & Sons, Inc. 1995-1999), in particular Chapters 10 and 16; and CURRENT PROTOCOLS IN PROTEIN SCIENCE Eds. Coligan et al., (John Wiley & Sons, Inc. 1995-1999), in particular Chapters 1, 5 and 6.

Various combinations of one or more additional agents as known by one of skill in the art for reducing, alleviating and/or preventing inflammation (and/or for treating or preventing a disease, disorder and/or condition associated with inflammation) may be administered to a subject in need thereof in addition to a therapeutically effective amount of one or more of the isolated proteins comprising an amino acid sequence according to SEQ ID NOS:1-31 (or a biologically active fragment or variant thereof). That is, one or more additional agents traditionally used for the treatment and/or prevention of inflammation may be administered to a subject in addition to a therapeutically effective amount of the isolated proteins comprising an amino acid sequence according to SEQ ID NOS:1-31 (or a biologically active fragment or variant thereof).

For example, nonsteroidal anti-inflammatory drugs (NSAIDs), aminosalicylates, corticosteroids, immunosuppressants, anti-cytokine/cytokine receptor agents (e.g., anti-TNFα agents, anti-IL-5 agents, anti-IL-13 agents, anti-IL-17 agents, and anti-IL-6R agents) particularly anti-cytokine/cytokine receptor antibodies, antibiotics, and combinations thereof can be administered with one or more isolated proteins comprising an amino acid sequence according to SEQ ID NOS:1-31 (or a biologically active fragment or variant thereof) in certain embodiments for reducing, alleviating and/or preventing inflammation.

In certain embodiments, the one or more additional agents provide a conserving effect on the one or more isolated proteins set forth in FIGS. 1 and 2, such as comprising an amino acid sequence according to SEQ ID NOS:1-31 (or a biologically active fragment or variant thereof). In further embodiments, the one or more isolated proteins comprising an amino acid sequence according to SEQ ID NOS:1-31 (or a biologically active fragment or variant thereof) provide a conserving effect on the one or more additional agents. In still further embodiments, the one or more additional agents provide a complimentary effect to the action of the one or more isolated proteins comprising an amino acid sequence according to SEQ ID NOS:1-31 (or a biologically active fragment or variant thereof), preferably eliminating or reducing the frequency or severity of (and/or preventing) one or more symptoms associated with inflammation.

As is well known to one of skill in the art, nonsteroidal anti-inflammatory drugs (NSAIDs), also referred to as nonsteroidal anti-inflammatory agents (NSAIAs), are drugs with analgesic, antipyretic and anti-inflammatory effects, and include salicylates (e.g., aspirin) and propionic acid derivatives (e.g., ibuprofen and naproxen.

Aminosalicylates are well known in the art for use in the treatment of inflammatory bowl disease (particularly ulcerative colitis), and include, for example, balsalazide, mesalazine, olsalazine, and sulfasalazine.

As is well known to one of skill in the art, corticosteroids are drugs that closely resemble cortisol, a hormone produced by the adrenal glands. Exemplary corticosteroids include, without limitation, cortisone, prednisone, prednisolone, and methylprednisolone.

Immunosuppressants are well known in the art for use in the treatment of inflammation associated with certain diseases or conditions, and include, for example, the drugs ciclosporin, azathioprine and mycophenolate.

As is well known to one of skill in the art, anti-cytokine/cytokine receptor agents (e.g., anti-TNFα agents, anti-IL-5 agents, anti-IL-13 agents, anti-IL-17 agents, and anti-IL-6R agents) include, without limitation, small molecule inhibitors and antibodies.

In some embodiments, the combination of one or more isolated proteins comprising an amino acid sequence according to SEQ ID NOS:1-31 (or a biologically active fragment or variant thereof) and one or more additional agents produces a synergistic effect in the treatment and/or prevention of inflammation. Accordingly, the present invention also includes a method of enhancing the therapeutic effectiveness of an agent in treating airy condition for which such agents are used (e.g., inflammation and any associated disease, disorder and/or condition).

In one embodiment, one or more isolated proteins in FIGS. 1 and/or 2, such as comprising an amino acid sequence according to SEQ ID NOS:1-31 (or a biologically active fragment or variant thereof) is administered prior to the administration of the one or more additional agents. In another embodiment, one or more isolated proteins of FIGS. 1 and/or 2 such as comprising an amino acid sequence according to SEQ ID NOS:1-31 (or a biologically active fragment or variant thereof) is administered after the administration of the one or more additional agents. In still another embodiment, one or more isolated proteins of FIGS. 1 and 2, such as comprising an amino acid sequence according to SEQ ID NOS:1-31 (or a biologically active fragment or variant thereof) is administered simultaneously with the administration of the one or more additional agents. In yet another embodiment, administration of one or more isolated proteins of FIGS. 1 and/or 2, such as comprising an amino acid sequence according to SEQ ID NOS:1-31 (or a biologically active fragment or variant thereof) and the administration of the one or more additional agents (either sequentially or concurrently) results in reduction or alleviation of inflammation that is greater than such reduction or alleviation from administration of either the one or more isolated proteins of FIGS. 1 and/or 2, such as comprising an amino acid sequence according to SEQ ID NOS:1-31 (or a biologically active fragment or variant thereof) or one or more additional agents in the absence of the other.

The one or more isolated proteins of FIGS. 1 and/or 2, such as comprising an amino acid sequence according to SEQ ID NOS:1-31 (or a biologically active fragment or variant thereof) and one or more additional agents can be administered by any conventional method/route available for use in conjunction with therapeutic compositions, as is well known to one of skill in the art. Such methods include, without limitation, administration by way of microneedle injection into specific tissue sites, such as described in U.S. Pat. No. 6,090,790, topical creams, lotions or sealant dressings applied to sites of inflammation, such as described in U.S. Pat. No. 6,054,122 or implants which release the one or more isolated proteins of FIGS. 1 and/or 2, such as comprising an amino acid sequence according to SEQ ID NOS:1-31 (or a biologically active fragment or variant thereof) such as described in International Publication WO 99/47070.

In this regard, compositions comprising one or more isolated proteins of FIGS. 1 and/or 2, such as comprising an amino acid sequence according to SEQ ID NOS:1-31 (or a biologically active fragment or variant thereof) and, optionally, one or more additional agents, may be administered in association with, or as a component of, a biomaterial, biopolymer, inorganic material such as hydroxyapatite or derivates thereof, surgical implant, prosthesis, wound dressing, compress, bandage, or the like suitably impregnated, coated or otherwise comprising the composition.

Suitably, the composition comprises an appropriate pharmaceutically-acceptable carrier, diluent or excipient.

Preferably, the pharmaceutically-acceptable carrier, diluent or excipient is suitable for administration to mammals, and more preferably, to humans.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates, and pyrogen-free water.

A useful reference describing pharmaceutically acceptable carriers, diluents and excipients is Remington's Pharmaceutical Sciences (Mack Publishing Co. NJ USA, 1991).

Any safe route of administration may be employed for providing a subject with compositions comprising one or more isolated proteins or FIGS. 1 and/or 2, such as comprising an amino acid sequence according to SEQ ID NOS:1-31 (or a biologically active fragment or variant thereof) and, optionally, one or more additional agents. For example, oral rectal, parenteral, sublingual, buccal, intravenous, intra-articular, intra-muscular, intra-dermal, subcutaneous, inhalational, intra-nasal, intraocular, intraperitoneal, intracerebroventricular, transdermal, and the like may be employed.

Dosage forms include tablets, dispersions, suspensions, injections, solutions, syrups, troches, capsules, suppositories, aerosols, transdermal patches, and the like. These dosage forms may also include injecting or implanting controlled releasing devices designed specifically for this purpose or other forms of implants modified to act additionally in this fashion. Controlled release of one or more isolated proteins of FIGS. 1 and/or 2, such as comprising an amino acid sequence according to SEQ ID NOS:1-31 (or a biologically active fragment or variant thereof) and, optionally, one or more additional agents, may be effected by coating the same, for example, with hydrophobic polymers including acrylic resins, waxes, higher aliphatic alcohols, polylactic and polyglycolic acids, and certain cellulose derivatives such as hydroxypropylmethyl cellulose. In addition, the controlled release may be affected by using other polymer matrices, liposomes and/or microspheres.

The above compositions may be administered in a manner compatible with the dosage formulation, and in such amount as is pharmaceutically/therapeutically-effective. The dose administered to a subject, in the context of the present invention, should be sufficient to effect a beneficial response (e.g., a reduction in inflammation) in a subject over an appropriate period of time. The quantity of one or more isolated proteins of FIGS. 1 and/or 2, such as comprising an amino acid sequence according to SEQ ID NOS:1-31 (or a biologically active fragment or variant thereof) to be administered may depend on the subject to be treated, inclusive of the age, sex, weight and general health condition thereof, factors that will depend on the judgment of a practitioner of ordinary skill in the art.

Compositions as described herein may also include expression vectors, such as viral vectors (e.g., vaccinia, adenovirus and adenovirus-associated viruses (AAV), retroviral and lentiviral vectors, and vectors derived from herpes simplex virus and cytomegalovirus. Gene therapy is also applicable in this regard, such as according to methods set forth in U.S. Pat. No. 5,929,040 and U.S. Pat. No. 5,962,427.

So that the invention may be readily understood and put into practical effect, the following non-limiting Examples are provided.

### EXAMPLES

### Materials & Methods

### Sequence Data, and Identification and Bioinformatic Analyses of TIMPs

The sequence data obtained from public sequence databases (i.e. National Center for Biotechnology Information at http://www.ncbi.nlm.nih.gov/; ENSEMBL Genome Browser at http://www.ensembl.org/index.html; WormBase, at www.wormbase.org; GeneDB at http://www.genedb.org/; www.gasserlab.org) [32-34,39,40,42-45] and analysed herein included known TIMP amino acid sequences from Homo sapiens (GenBank accession numbers XP_010392.1, NP_003246.1, P35625.1 and Q99727.1), Mus musculus (accession numbers P12032.2, P25785.2, P39876.1 and Q9JHB3.1), Canis familiaris (AF112115.1), Gallus gallus (AAB69168.1), Oryctolagus cuniculus (AAB35920.1), Drosophila melanogaster (AAL39356.1), A. caninum (AF372651.1 and EU523698.1), A. duodenale (ABP88131.1) and Caenorhabditis elegans (NP_505113.1), as well as predicted peptides inferred from (i) the whole or draft genome sequences of S. mansoni, S. japonicum, S. haematobium (www.genedb.org), A. suum (www.wormbase.org), T. spiralis (http://www.ncbi.nlm.nih.gov/nuccore/316979833), Brugia malayi and Wuchereria bancrofti (human filarial nematodes) (http://www.sanger.ac.uk/; [46]), N. americanus (human hookworm; [36]), and (ii) the transcriptomes of T. suis (swine whipworm), Oesophagostomum dentatum (swine nodule worm) (http://www.gasserlab.org), Dictyocaulus filaria (sheep lungworm, [47]) and C. sinensis, O. viverrini (human liver flukes), Fasciola hepatica and F. gigantica (bovine and deer liver fluke, respectively) (http://www.gasserlab.org).

The algorithms BLASTp [48] and InterProScan [49] were used to identify TIMP proteins in each of the genomic and transcriptomic datasets based on sequence homology (e-value cut-off: 10-5) with known TIMP proteins from eukaryotes [50]. In addition, the software pScan (http://www.psc.edu/general/software/packages/emboss/appgroups/pscan.html) was used to identify regular expression based diagnostic patterns for TIMPs (Prosite: PS00288). Signal peptides were also predicted using the program SignalP 3.0, employing both the neural network and hidden Markov Models [51]. Putative ES TIMP proteins were identified based on the presence of a signal peptide and sequence homology to one or more known ES proteins listed in the Secreted Protein (http://spd.cbi.pku.edu.cn/; [52]) and the Signal Peptide (http://proline.bic.nus.edu.sg/spdb/index.html; [53]) databases.

### Secondary Structure Predictions and Homology Modeling

Structure-based sequence alignments of TIMP proteins were computed and manually edited with SBAL [54] guided by secondary structure elements predicted using the PSIPRED software [55]. Individual structure-based alignments of amino acid sequences were subjected to analysis by Bayesian inference (BI) using the program MrBayes v.3.1.2 [56] and verified by Maximum Likelihood analysis using the program MEGA v.5 [57] and the Jones-Taylor-Thornton substitution model with uniform rates among sites (JTT+G+I). Each BI analysis was conducted for 1,000,000 generations (ngen=1,000,000) with every 100-th tree being saved, using the following parameters: rates=gamma, aamodelpr=mixed, and the other parameters left at the default settings. Tree and branch lengths were measured employing the parameter ‘sumt burnin=1000; an unrooted, consensus tree was constructed, with ‘contype=halfcampat’ nodal support being determined using consensus posterior probabilities and displayed employing the software FigTree (http://tree.bio.ed.ac.uk/software/figtree/). For selected TIMPs, homologues with known three-dimensional structures were identified using the protein-fold recognition software pGenTHREADER [58] and selected as templates for comparative modeling using MODELLER [59]. Twenty independent models were generated, and the model with the lowest energy was selected, its geometry analysed using PROCHECK [60] and then inspected visually with PyMOL [61].

### Assessment of Levels of Transcription of TIMP-Encoding Genes

The raw sequence reads derived from each of the nonnormalized cDNA libraries from A. suum infective L3s (iL3s; from eggs), migrating L3s (from liver and lung), fourth-stage larvae (L4s, from the small intestine) and muscular and reproductive tissues from each adult male and female [34], N. americanus iL3s and adults (mixed males and females) [36], as well as S. haematobium eggs and adult male and female [40] were mapped to the longest contigs encoding individual putative TIMP proteins using the program SOAP2 [62]. Briefly, raw sequence reads were aligned to the non-redundant transcriptomic data, such that each raw sequence read was uniquely mapped (i.e. to a unique transcript). Reads that mapped to more than one transcript (designated ‘multi-reads’) were randomly assigned to a unique transcript, such that they were recorded only once. To provide a relative assessment of transcript abundance, the number of raw reads that mapped to each sequence was normalized for length (i.e. reads per kilobase per million reads, RPKM) [34,40,63].

### Results & Discussion

### TIMP Proteins of Parasitic Helminths

A total number of 15 protein sequences with high homology (e-value cut-off: 10-5) to known eukaryotic TIMPs were predicted from the complement of sequence data available for parasitic helminths (Table 1), thus representing a solid resource for future structural and functional investigations of this protein family in parasites. The sequence data in FASTA format analysed in the present article is available in Additional file 1. Of the datasets included here, the complement of protein coding genes available for N. americanus and A. suum encoded the largest number of predicted TIMP proteins (n=8 and 3, respectively; cf. Table 1). Three N. americanus (i.e. NECAME_13168, NECAME_07191 and NECAME_08458; cf. Table 1) and all A. suum TIMPs (GS_21732, GS_04796 and GS_08199; cf. Table 1) were predicted to contain an N-terminal signal peptide, in accordance with previous observations of A. caninum Ac-TMP-1 and Ac-TMP-2 and a netrin-domain containing homologue from Ancylostoma ceylanicum (=excretory-secretory protein 2, AceES-2), respectively [25-27,64]. Despite the sequence similarities between Ac-TMP-1, Ac-TMP-2 and AceES-2, the latter did not display human MMP inhibitory activity in vitro, thus suggesting a different function of this protein in vivo [64]. However, it should be noted that the partial MMP inhibitory activity of Ac-TMP-2 described by Zhan et al. [26] was based on a vast molar excess of recombinant TMP-2, well beyond the 1:1 inhibitor:enzyme molar ratio required for inhibition of mammalian MMPs by their TIMP counterparts [23].

Moreover, TIMPs seem to require the C-X-C motif at the N-terminus to allow insertion into the MMP active site cleft and subsequent inhibition of catalytic activity; recombinant Ac-TMP-2 was engineered to contain a long N-terminal extension donated by the plasmid vector, so it is premature to unequivocally assign MMP inhibitory activity to the hookworm TIMPs without further work. In A. ceylanicum, secretion of AceES-2 begins soon after infection of the experimental hamster host, and steadily increases in correspondence with the onset of blood-feeding activity [65]. Furthermore, a single oral dose of recombinant AceES-2 resulted in reduced anaemia following challenge infection of hamsters with A. ceylanicum [66], which led to speculations that this molecule may play a role in the pathogenesis of hookworm disease [66]. A role for hookworm TIMPs in molecular processes linked to the invasion of the mammalian hosts and/or the inhibition of hosts MMPs at the final site of attachment has also been hypothesized, based on the fact that Ac-TMP-2 could be isolated solely from extracts and ES products of A. caninum adults, despite the corresponding mRNA being detected from both L3s and adults of this parasite [26].

Of the eight genes encoding putative TIMPs in N. americanus, transcription of NECAME_13168 and NECAME_07191 was significantly up-regulated in iL3s (cf. Table 1; [36]), thus supporting a role for these proteins in the infection process of the human host. Conversely, NECAME_08457 and NECAME_08458 displayed high transcription levels in adult N. americanus (cf. Table 1; [36]), which likely reflects a diversification of function of members of this protein family in different developmental stages of this parasite. In the future, studies of differential transcription of genes encoding TIMPs in both genders and different tissues of N. americanus may help elucidate the roles that these molecules play in the fundamental molecular biology of the adult nematode. In A. suum, transcription of GS_04796 was significantly up-regulated in the adult female reproductive tissue of this nematode, whereas GS_21732 was up-regulated in the male muscle (cf. Table 1; cf. [34]).

The putative TIMP proteins encoded by GS_04796 and GS_21732 share ˜40% similarity with C. elegans CRI-2 (WBGene00019478; http://www.wormbase.org), the expression of which has been localized to the body wall musculature and to the vulval, anal and pharyngeal muscles of the adult nematode (cf. htpp://www.wormbase.org). In C. elegans, cri-2 is known to function in the cascade of molecular events linked to the regulation of the innate immune response to lipopolysaccharide (LPS) [67]. In a previous study, inhibition by small interfering RNAs (siRNAs) of the M. musculus ortholog of C. elegans cri-2 in a mouse macrophage cell line stimulated with Escherichia coli LPS resulted in decreased production of interleukin-6 (IL-6) [67]. This cytokine, in vivo, is associated with a wide range of biological activities, which include the generation of acute-phase reactions in response to infections by pathogens [68].

In flatworms, the S. haematobium gene A_01727 encoded the only trematode TIMP protein that could be identified using computational methods. Analysis of transcriptional regulation of S. haematobium A_01727 in different developmental stages revealed that this molecule is up-regulated in the adult male of this parasitic trematode (Table 1; cf. [40]). The transcript encoding mouse TIMP-1 is up-regulated in male gonads during testis morphogenesis, while expression of the corresponding protein was restricted to the cords of foetal testes [70]. In addition, the human and mouse genes encoding TIMP-2 are known to include the differential display clone 8 (DDC8) gene, whose transcription is enhanced during spermatogenesis [71]. These observations, together with earlier findings of increased expression of TIMP-1 in human foetal Sertoli cells [72,73] and testicular expression of TIMP-2 in rats [74], led to the hypothesis that these molecules may play specific roles during testis organogenesis and development [70], as well as in the migration of germ cells through the seminiferous epithelium [71]. Therefore, it is tempting to speculate a role for S. haematobium A_01727 in biological processes linked to the reproductive activity of the adult male fluke; however, this hypothesis requires rigorous testing. In the future, genetic manipulation of N. americanus, A. suum and S. haematobium by RNA interference (RNAi) and/or transgenesis [75-78], may help elucidate the functions of putative helminth TIMPs in the reproductive biology of these organisms, as well as in other fundamental molecular processes, for instance those linked to host invasion and modulation of the host's innate immune response.

Genomic sequence data with identity to S. haematobium A_01727 were detected in both S. mansoni (Smp_087690; e-value 3e-110) and S. japonicum (Sjp_0053050.1; e-value 6.3e-64). However, the sequence overlap between the amino acid sequence predicted from S. haematobium A_01727 and the corresponding homologues from S. mansoni and S. japonicum was limited to the NTR N terminal module (cf. FIG. 2), which would make any inference of the presence of TIMP-encoding genes in the genome sequences of the latter two species highly speculative. While it is possible that fragmentation of the Open Reading Frames (ORFs) of TIMP-encoding genes in the current assemblies of the S. mansoni and S. japonicum genomes might have occurred, the absence of homologues of eukaryote TIMPs in other species whose whole-genome sequences are currently available (e.g. B. malayi and T. spiralis) may reflect the substantial variations, both in sequence and in length, among members of this protein family in helminths [23]. Indeed, a search of the characteristic features of the N-terminal NTR module of eukaryote TIMPs using the PScan software revealed the presence of members of the netrin protein family in all parasitic helminths analysed herein (n=26; range 1-5; cf. Table 1). This finding is in accordance with current knowledge that the genomes of helminths encode single-domain TIMP proteins that are homologous to the N-terminal domain of vertebrate TIMPs, while lacking the corresponding C-terminal region [79]. In eukaryotes, the N-terminal NTR domain of TIMPs is known to be responsible for their metalloprotease inhibitory activity [24,80,81], whereas the C-terminal domain provides binding sites for the metalloproteases [80,82,83] or for binding TIMPs to the cell surface and/or the extracellular matrix [24,81,84]. When separated from the corresponding C-terminus, the N-terminal domain of TIMPs retains its metalloprotease inhibitory activity [24,81-84]. While, based on this knowledge, single-domain helminth TIMPs may be hypothesized to exert similar metalloprotease inhibitory activities as their vertebrate counterparts, the amino acid residues present at position 2 of some mature helminth molecules (e.g. lysine, arginine and glutamine; cf. FIG. 2) are atypical for vertebrate TIMPs and suggest that these proteins may perform functions that are unrelated to the inhibition of metalloprotease activity (see [23,85]). Comparative structural analyses of the amino acid sequences of TIMP proteins, as well as the N-terminal NTR module are essential to assist in-depth investigations of the functions of this family of helminth proteins.

### Structural Analyses of Eukaryote TIMPs

Structurally, the four human TIMPs are well characterized (cf. http://www.rcsb.org). These proteins consist of two domains, an N-terminal domain (N-TIMP) adopting the NTR fold, and a C-terminal domain (C-TIMP). Tertiary structures of full-length TIMP-1, TIMP-2, as well as NTIMP-1, N-TIMP-2 and N-TIMP-3 have been determined, some in complex with their target MMPs (for an overview, see Table 2). Both N-TIMP and C-TIMP are internally stabilised by three intra-domain disulphide bridges and their structural elements are not intertwined, suggesting that the two moieties are indeed individual folding units, i.e. domains. This notion is further supported by the observation that N-TIMPs can be obtained as folded entities in vitro that display MMP inhibitory activity [79,86-88].

The shape of full-length TIMPs appears wedge-like, and the extreme N-terminus is responsible for the inhibitory action of MMPs by interaction with the protease active site cleft. In some instances, additional interactions have been observed between C-TIMP and peripheral areas of the protease that are distant to the catalytic site. However, in the case of the TIMP-2/MMP-2 complex, the interaction of C-TIMP-2 and the hemopexin domain of MMP-2 significantly enhances the affinity of the inhibitor [89,90]. The main interactions of TIMPs with their target proteases are formed by a continuous peptide at the N terminal end (Cys1-Pro5 in human TIMP-1) and in a loop connecting two adjacent β-strands (Met66-Cys70 in human TIMP-1). The two regions are covalently linked by a disulphide bond (Cys1-Cys70 in human TIMP-1), and are located in the netrin module (N-TIMP) of the protein which adopts the fold of a five-stranded α-barrel with Greek key topology (OB-fold) flanked by two α-helices.

The N-terminus of N-TIMP inserts into the active site of the target protease and the α-amino and the carbonyl group of Cys-1 (human TIMP-1) coordinate the active site zinc ion of the protease by displacing a water molecule otherwise bound to the metal [23]. Residue 2 (Ser, Thr) projects into the specificity (S1) pocket of the protease. Residues 3-5 interact with the protease residues in the primed subsites, which normally harbour substrate residues C-terminal of the scissile bond. Similarly, residues 66-70 of TIMP-1 occupy the non-primed subsites of the protease that otherwise interact with the residues N-terminal to the scissile bond. As apparent from the structure-based amino acid sequence alignment (FIG. 2), TIMPs from parasitic helminths are characterised by higher sequence variation than their mammalian homologues, in accordance with the results of previous analyses of invertebrate TIMPs [23]. With respect to structure-function relationships, however, the most important feature grafted onto the netrin fold seems to be the conformation neighbouring Cys-1. In vertebrate TIMPs, 2 is either a serine or threonine that projects into the protease specificity pocket. It is important to note that neither Ac-TMP-1 nor Ac-TMP-2 have been convincingly shown (via 1:1 inhibitor:enzyme molar ratios) to possess MMP inhibitory activity. Moreover, AceES-2 produced with a flush N-terminus was screened for MMP activity at 15:1 and 115:1 molar ratios and did not display inhibitory activity (cf. [64]). The amino acid sequence alignment in FIG. 2 highlights the general motif of TIMPs, C-X-C, in this region. It shows for the helminth TIMP with published inhibitory activity, Ac-TMP-2, that in addition to serine and threonine, lysine is a tolerated residue at position 2 for inhibition. Notably, AceES-2 and Ad-TIMP-1 from A. duodenale lack the second cysteine residue as well as a suitable residue at position 2 (Ser/Thr/Lys) able to protrude into the S1′ pocket of the protease for inhibition (cf. FIG. 2).

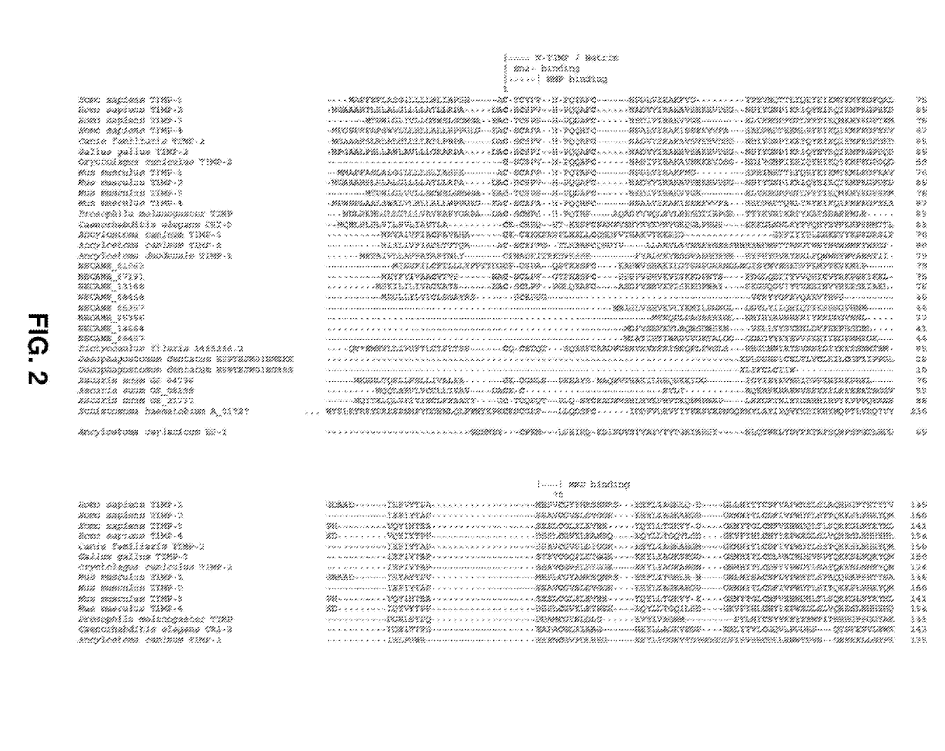

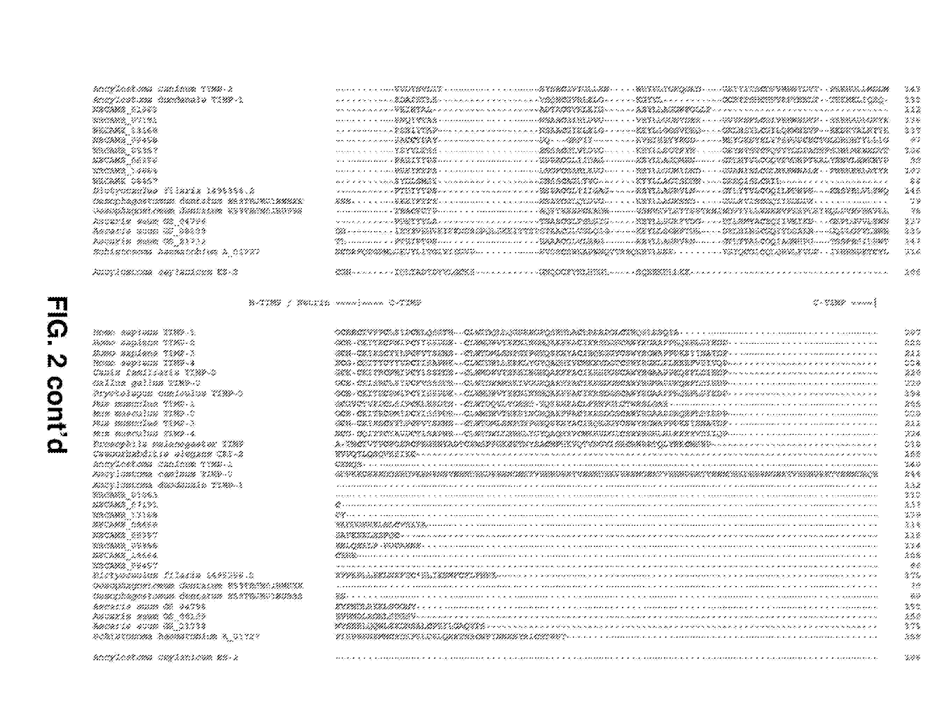

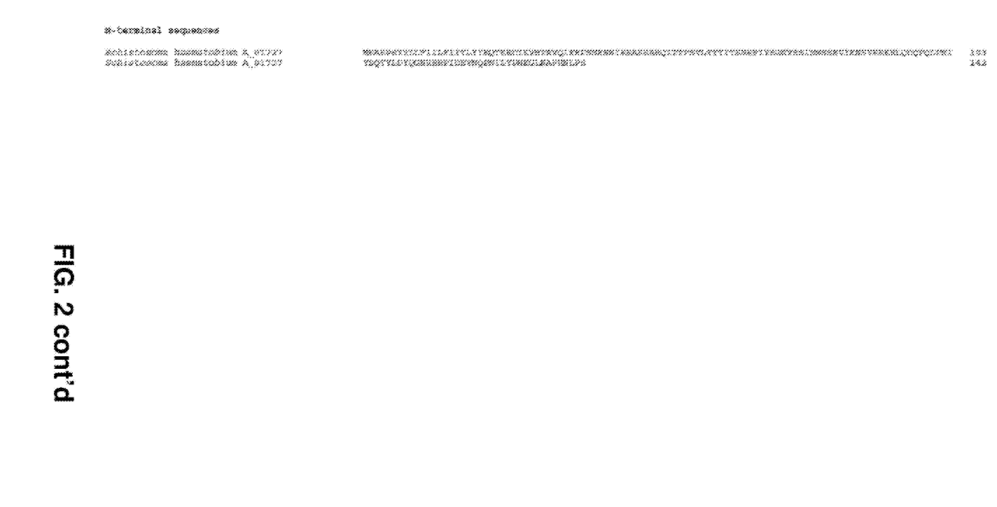

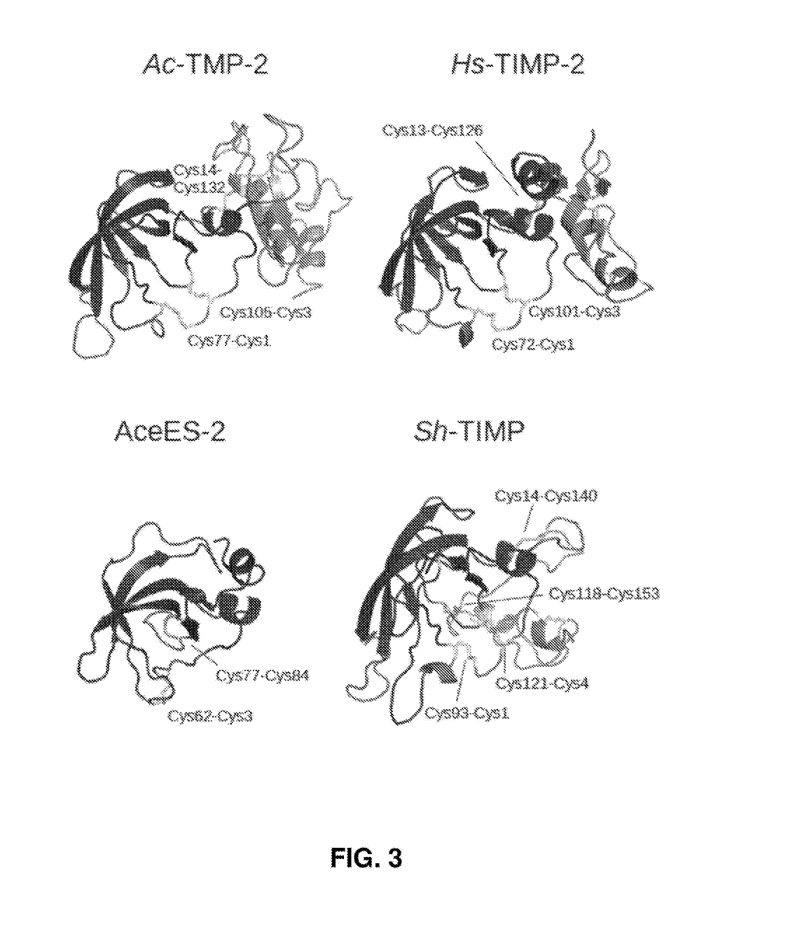

On this basis, one would predict Ad-TIMP-1 to not have any MMP-inhibitory activity. Thus, helminth TIMPs that show conservation at position 2 are likely to display inhibitory activities against human MMPs. The S. haematobium protein encoded by A_01727 possesses two residues (Arg-Ser) between the two N-terminal cysteine residues, which makes the prediction of functional effects difficult in the absence of experimental structures. Helminth TIMPs for which complete amino acid sequence data is available, with the exception of Ad-TIMP-1, show conservation of the crucial structural elements of the NTR module, such as the two N-terminal cysteine residues and their covalent binding partners, as well as residues relevant for maintaining the OB-fold. The areas of largest variation are three surface-exposed loop areas, namely residues 28-41,56-59 and 66-70 (Hs-TIMP-2 numbering; see FIG. 2). Notably, there is high conservation of a basic residue (Arg 20 in Hs-TIMP-1) in vertebrate and helminth TIMPs, which is an exposed residue on the surface distal to the protease interaction site (FIG. 3). To our knowledge, a physiologically important function for this residue is yet to be described. Its location (at the surface of the protein) suggests a protein-protein or protein-matrix interaction; however, basic residues at this position have not been reported to be involved in extra-cellular matrix binding [91]. While S. haematobium A_01727 shares the lowest amino acid sequence identity with the other eukaryote TIMPs (cf. FIG. 2), the structure-based sequence alignment, together with the accordingly predicted 3D structure, indicate, that it may be a functional member of the TIMP family of proteins. This conclusion is based on the presence of all conserved cysteine residues required for intramolecular disulphide bonds of a netrin-like fold, as well as conservation of the serine residue (Ser3) expected to protrude into the catalytic site of an MMP.

### Phylogenetic Analysis

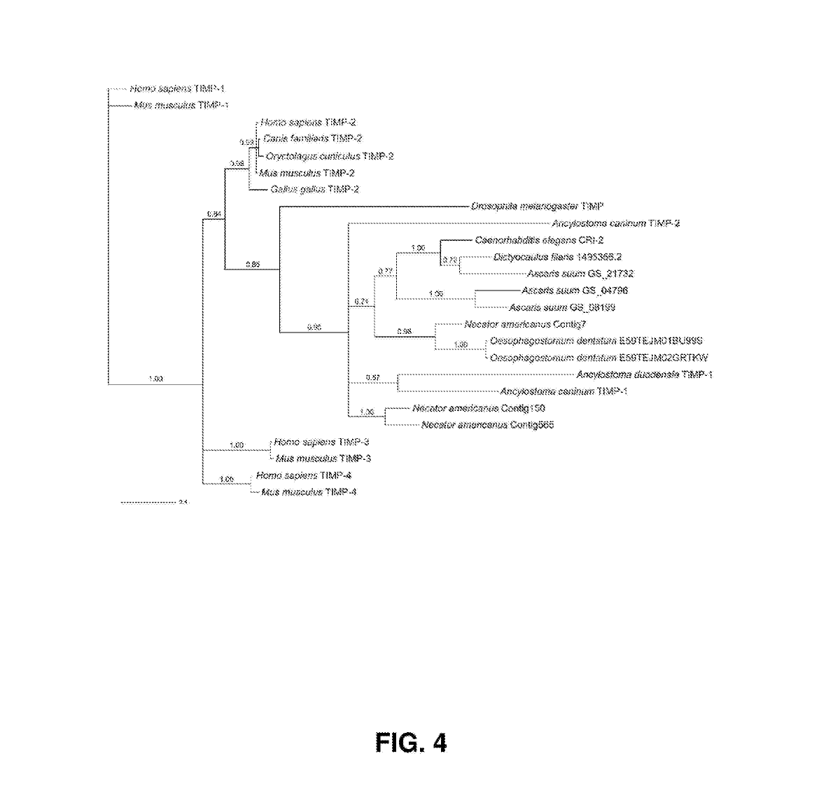

The phylogenetic analysis of eukaryote TIMPs allowed us to study the relationships between helminth TIMPs and their vertebrate counterparts (FIG. 4). The analysis identified one main clade comprising TIMPs from invertebrates, including free-living and parasitic helminths (nodal support: 0.90), to the exclusion of clades formed by homologues from vertebrates (cf. FIG. 4). Within the invertebrate clade, a sub-clade representing TIMPs from nematodes clustered to the exclusion of the TIMP protein from D. melanogaster (nodal support: 0.76; cf. FIG. 4), supporting the existence of a monophyletic group of TIMPs for parasitic nematodes. Following the inclusion of S. haematobium A_01727 in the phylogenetic analysis, the monophyly of the nematode TIMP clade with respect to the vertebrate homologues was maintained. No distinct separation between TIMPs from hookworms and those from other free-living and parasitic nematodes was observed, thus supporting the hypothesis that nematode TIMPs may be characterised by specific functional properties, distinct from those of their vertebrate homologues. Whether nematode TIMPs have originated following loss of the C-terminal domain from a vertebrate ancestor or from a distinct gene line (cf. [23]) remains to be explored.

TIMP protein amino acid sequences designated as SEQ ID NOS:1-31 and shown in FIG. 1 and/or FIG. 2 may have anti-inflammatory properties suitable for prevention or treatment of inflammatory conditions. In previous work that has been described in PCT/AU2013/000247 published as WO2013/134822, AcTIMP-1 (SEQ ID NO:32) and AcTIMP-2 (SEQ ID NO:33) were shown to have anti-inflammatory activity. In initial studies, recombinant Ac-TMP-1 (SEQ ID NO:32) and Ac-TMP-2 (SEQ ID NO:33) afforded excellent protection against weight loss in two separate TNBS colitis experiments. Ac-TMP-2 was further assessed for clinical and macroscopic scores and colon length and in this regard afforded significant reduction in intestinal pathology. Furthermore, treated mice exhibited a significantly reduced eosinophilia, perivascular and peribronchial cellular infiltration of the lungs. Compared to the naïve group, PBS-treated BSA-challenged mice exhibited increased levels of Th2 cytokines such as interleukin (IL)-5 and IL-13, as well as markers of inflammation such as IL-6. It was found that Ac-TMP-1 treatments resulted in one- to five-fold less (respectively) IL-5, and 2-fold less IL-13 in the lungs. Inflammatory cytokine IL-6 was also 2 to 3-fold decreased in mice treated with Ac-TMP-1. While pro-inflammatory cytokines TNFα or IFNγ are not directly associated with asthma-induced inflammation, levels were 3- and 5-fold increased (respectively) in treated mice. However, levels remained unaffected by the Ac-TMP-1 treatment, suggesting that Ac-TMP-1 is well tolerized and does not induce inflammatory responses. IL-12 and MCP-1 levels remained unaffected by the Ac-TMP-1 treatment, meaning that the prevention of BSA-induced inflammation does not require the induction of Th1 response and does not affect monocyte chemotaxis. Surprisingly, Ac-TMP-1 treatment induced a 2 to 3-fold decrease in IL-17A levels in the lungs, which in high levels has been reported to be associated with severe asthma-induced inflammation and airway hyper-responsiveness. Taken together, these results illustrated that Ac-TMP-1 reduce significantly BSA-induced airway infiltration of eosinophils and lymphocytes, but also Th2 and Th17 responses, as well as pro-inflammatory cytokines such as IL-6.

In further experiments investigating asthma, mice treated with Ac-TMP-1 (SEQ ID NO:32) or Ac-TMP-2 (SEQ ID NO:33) showed a significantly decreased eosinophilia in the airways as compared to the mock injection group, there was no infiltration of eosinophils in the peritoneum, indicating that Ac-TMP-1 and Ac-TMP-2 prevent the induction of eosinophils at sites of allergic or inflammatory response only.

Lung cells from OVA-challenged mice demonstrated increased levels of IL-5, IL-10 and IL-13 secretion with OVA stimulation in vitro. Supernatant levels of MCP-1 and IL-17A, on the other hand, were similarly elevated in both PBS-mock and OVA-challenged mouse lung cells when stimulated with OVA. In accordance with the bronchoalveolar lavage findings, levels of Th2 cytokines, IL-5, IL-10 and IL-13, and the pro-inflammatory cytokines, MCP-1 and IL-17A, were reduced in the OVA-stimulated lung cells from Ac-TMP-1 treated mice. Similarly, lung cytokine content was significantly decreased in mice treated with Ac-TMP-2 suggesting that Ac-TMP-2 efficiently suppresses Th2 and pro-inflammatory cytokines such as IL-6 and IL-17A.

To assess whether Ac-TMP-2, either administered only during the first OVA challenge (+/−) or during both sets of OVA challenges (+/+), decreased airway inflammation in a mouse model of chronic asthma, bronchoalveolar lavages of naïve mice, mice treated with PBS-mock injections, or mice treated with Ac-TMP-2 (+/− and +/+) were collected and analysed by FACS. Regardless of whether Ac-TMP-2 was administered during the first challenge (+/−) or both challenges (+/+), treated mice demonstrated a significant reduction in both total cellular and eosinophilic airway infiltration when compared to mice treated with PBS-mock injections, in this model of OVA-induced chronic asthma.

To determine whether Ac-TMP-2 administered locally via intranasal injections could attenuate airway inflammation when given in a preventative (Tp, before the OVA-challenge) or a curative (Tc, after the OVA-challenge) manner, bronchoalveolar lavages of naïve mice, OVA-challenged mice treated with PBS-mock injections, or OVA-challenged mice treated with Ac-TMP-2 (Tc and Tp) were collected and analysed by FACS from which total and differential cell counts were derived. Regardless of whether mice were treated in a preventative or curative fashion, intranasal Ac-TMP-2 significantly attenuated both total and eosinophilic airway cellular infiltration. Importantly, these data highlighted that Ac-TMP-2 may also be administered locally and not just parenterally to prevent airway inflammation in this murine model of asthma.

Whole protein extracts from the lungs of naïve mice, OVA-challenged mice treated with PBS-mock injections, or OVA-challenged mice treated with Ac-TMP-2 (Tc and Tp) were prepared and analysed for the Th2 cytokines, IL-5 and IL-13, by Cytometric Bead Array (CBA). Compared to the naïve group, PBS-treated OVA-challenged mice demonstrated significantly elevated levels of both IL-5 and IL-13. We found that treatment with Ac-TMP-2, in either a preventative or curative manner, significantly reduced IL-5 and IL-13 levels. Taken together these findings illustrated that Ac-TMP-2 when administered intranasally significantly reduced OVA-induced eosinophilic airway infiltration and the associated Th2 inflammatory response.

The administration of Ac-TMP-2 to naïve mice significantly induced the recruitment of Tregs into the lamina propria of the small intestine. Conversely, a significant decrease in the frequency of Tregs from the mesenteric lymph nodes (MLN) was observed with Ac-TMP-2 treatment. These data suggest a migration pattern of Tregs from the MLN towards the mucosa of the intestine. In support of this, sixty percent of the lamina propria Tregs expressed the chemokine receptor CCR9, indicating that they have been imprinted in the gut-associated draining lymph nodes (i.e. MLN). This observation coincides with data suggesting that Tregs generated in the MLN accumulate in the mucosa in order to maintain tolerance to ubiquitous antigens.

Ac-TMP-2 treatment significantly reduced airway inflammation in OVA-challenged wild-type mice. Conversely, OVA-challenged DEREG mice treated with Ac-TMP-2 demonstrated comparable levels of bronchoalveolar infiltration to untreated DEREG mice challenged with OVA. In keeping with these findings, levels of the Th2 cytokines, IL-5, IL-10 and IL-13, and the pro-inflammatory IL-6 were significantly reduced in OVA-challenged wild-type mice, but not OVA-challenged DEREG mice, upon treatment with Ac-TMP-2. Taken together, these results suggested that Tregs play an integral role m the anti-inflammatory action of Ac-TMP-2 in this mouse model of asthma.

At least some of the aforementioned experimental methods, models and approaches will be utilized to experimentally confirm the anti-inflammatory activity of one or more of the proteins set forth in FIGS. 1 and/or 2, such as set forth in SEQ ID NOS:1-31. Initially, NECAME_07191, NECAME_13168 and Ancylostoma duodenale TIMP-1 will be tested in a TNBS experimental colitis model.

It is therefore expected that experimental verification will confirm that proteins comprising the amino acid sequences set forth in FIGS. 1 and/or 2, such as according to SEQ ID NOS:1-31, may have anti-inflammatory activity and accordingly be useful in the treatment or prevention of diseases or conditions including but not limited to asthma, asthma, emphysema, chronic bronchitis, and chronic obstructive pulmonary disease (COPD), Addison's disease, alkylosing spondylitis, celiac disease, chronic inflammatory demyelinating polyneuropathy (CIDP), chronic recurrent multifocal ostomyelitis (CRMO), Crohn's disease, demyelinating neuropathies, glomerulonephritis, Goodpasture's syndrome, Graves' disease, Guillain-Barre syndrome, Hashimoto's encephalitis, Hashimoto's thyroiditis, hypogammaglobutinemia, idiopathic thrombocytopenic purpura (ITP), insulin-dependent diabetes (type1), juvenile arthritis, Kawasaki syndrome, multiple sclerosis, myasthenia gravis, postmyocardial infarction syndrome, primary biliary cirrhosis, psoriasis, idiopathic pulmonary fibrosis, Reiter's syndrome, rheumatoid arthritis, sarcoidosis, scleroderma, Sjogren's syndrome, systemic lupus erythematosus (SLE), thrombocytopenic purpura (TTP), ulcerative colitis, vasculitis, vitiligo, and Wegener's granulomatosis.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein is incorporated herein by reference.

TABLE 1
Number of tissue inhibitor of metalloproteases (TIMP) and
netrin module(NTR)-containing protein sequences, respectively,
identified in each sequence dataset and listed according to taxa.
The number of proteins containing a predicted N-terminal
signal peptide (SP) is also indicated.

| | | NTR-module |
|---|---|---|
| | TIMPs | containing proteins |
| | (no. with SP) | (no. with SP) |
| Nematodes | | |
| Ascaris Suum | 3* (3) | 2 (—) |
| Brugia malayi | — | 1 (1) |
| Dictyocaulus filaria | 1 (1) | 1 (—) |
| Necator americanus | 3 (3) | 2 (—) |
| Oesophagostomum dentatum | 2 (2) | 1 (—) |
| Trichinella spiralis | — | 1 (1) |
| Trichuris suis | — | 2 (—) |
| Wuchereria bancrofti | — | 2 (—) |
| Trematodes | | |
| Clonorchis sinensis | — | 1 (—) |
| Fasciola gigantica | — | 2 (—) |
| Fasciola hepatica | — | 1 (—) |
| Opisthorchis viverrini | — | 4 (1) |
| Schistosoma haematobium | 1** (1) | 5 (—) |
| Schistosoma japonicum | — | 1 (—) |
| Schistosoma mansoni | — | 1 (—) |
| Total | 10 (10) | 26 (3) |

*Of these, As-GS_21732 was up-regulated in the muscular tissue of adult male.
*Sh-A_01727 was up-regulated in the adult male.

TABLE 2
Three dimensional strucures of tissue inhibitors of metalloproteases
(TIMPS) and their complexes available in the protein databank (PDB;
http://www.rcsb.org.pdb/home/home.do) as of November 2012.

| Protein | PDB Accession Code |
|---|---|
| N-TIMP-1 | 1d2b |
| MMP1:TIMP-1 | 2j0t |
| MMP3:TIMP-1 | 1uea |
| MMP3N:TIMP-1 | 1oo9 |
| MMP10:TIMP-1 | 3v96 |
| MMP14:TIMP-1 | 3ma2 |
| TIMP-2 | 1br9 |
| N-TIMP-2 | 2tmp |
| pro-MMP2-TMP-2 | 1gxd |
| MMP-13:TIMP-2 | 2e2d |
| MMP-14:TIMP-2 | 1bqq |
| MMP-14:TIMP-2 | 1buv |
| TACE:N-TIMP-3 | 3cki |

### REFERENCES

1. Hotez P J, Brindley P J, Bethony J M, King C H, Pearce E J, et al: Helminth infections: the great neglected tropical diseases. J Clin Invest 2008, 118:1311-1321.
2. Lustigman S, Prichard R K, Gazzinelli A, Grant W N, Boatin B A, et al: A research agenda for helminth diseases of humans: the problem of helminthiasis. PLoS Negl Trop Dis 2012, 6:e1582.
3. Rollinson D: A wake up call for urinary schistosomiasis: reconciling research effort with public health importance. Parasitology 2009, 136:1593-1610.
4. Shin H R, Oh J K, Masuyer E, Curado M P, Bouvard V, et al: Epidemiology of cholangiocarcinoma: an update focusing on risk factors. Cancer Sci 2010, 101:579-585.
5. Fried B, Reddy A. Mayer D: Helminths in human carcinogenesis. Cancer Lett 2011, 305:239-249.
6. Keiser J, Utzinger J: The drugs we have and the drugs we need against major helminth infections. Adv Parasitol 2010, 73:197-230.
7. Caffrey C R, Secor W E: Schistosomiasis: from drug deployment to drug development. Curr Opin Infect Dis 2011, 24:410-417.
8. Wolstenholme A J, Fairweather I, Prichard R, von Samson-Himmelstjerna G, Sangster N C: Drug resistance in veterinary helminths. Trends Parasitol 2004, 20:469-476.
9. Beech R N, Skuce P, Bartley D J, Martin R J, Prichard R K, et al: Anthelmintic resistance: markers for resistance, or susceptibility? Parasitology 2011, 138:160-174.
10. Hoste H, Torres-Acosta J F: Non chemical control of helminths in ruminants: adapting solutions for changing worms in a changing world. Vet Parasitol 2011, 180:144-154.
11. De Clercq D, Sacko M, Behnke J, Gilbert F, Dorny P, et al: Failure of mebendazole in treatment of human hookworm infections in the southern region of Mali. Am J Trop Med Hyg 1997, 57:25-30.
12. Reynoldson J A, Behnke J M, Pallant L J, Macnish M G, Gilbert F, et al: Failure of pyrantel in treatment of human hookworm infections (Ancylostoma duodenale) in the Kimberley region of north west Australia. Acta Trop 1997, 68:301-312.
13. Sacko M, De Clercq D, Behnke J M, Gilbert F S, Dorny P, et al: Comparison of the efficacy of mebendazole, albendazole and pyrantel in treatment of human hookworm infections in the southern region of Mali, West Africa. Trans R Soc Trop Med Hyg 1999, 93:195-203.
14. Albonico M, Engels D, Savioli L: Monitoring drug efficacy and early detection of drug resistance in human soil-transmitted nematodes: a pressing public health agenda for helminth control. Int J Parasitol 2004, 34:1205-1210.
15. Kotze A C, Kopp S R: The potential impact of density dependent fecundity on the use of the faecal egg count reduction test for detecting drug resistance in human hookworms. PLoS Negl Trop Dis 2008. 2:e297.
16. Gilleard J S, Beech R N: Population genetics of anthelmintic resistance in parasitic nematodes. Parasitology 2007, 134:1133-1147.
17. Kotze A C: Target-based and whole-worm screening approaches to anthelmitic discovery. Vet Parasitol 2012, 186:118-123.
18. Knox D P: Proteinase inhibitors and helminth parasite infection. Parasite Immunol 2007, 29:57-71.
19. Klotz C, Ziegler T, Danilowicz-Luebert E, Hartmann S: Cystatins of parasitic organisms. Adv Exp Med Biol 2011, 712:208-221.
20. Cappello M, Vlasuk G P, Bergum P W, Huang S, Hotez P J: Ancylostoma caninum anticoagulant peptide: a hookworm-derived inhibitor of human anticoagulant factor Xa. Proc Natl Acad Sci USA 1995, 92:6152-6156.
21. Molehin A J, Gobert G N, McManus D P: Serine protease inhibitors of parasitic helminths. Parasitology 2012, 139:681-695.
22. Hartmann S, Lucius R: Modulation of host immune responses by nematode cystatins. Int J Parasitol 2003, 33:1291-1302.
23. Brew K, Nagase H: The tissue inhibitors of metalloproteinases (TIMPs): an ancient family with structural and functional diversity. Biochim Biophys Acta 2010, 1803:55-71.
24. Bányai L, Patthy L: The NTR module: domains of netrins, secreted frizzled related proteins, and type I procollagen C-proteinase enhancer protein are homologous with tissue inhibitors of metalloproteases. Protein Sci 1999, 81636-1642.
25. Zhan B, Badamchian M, Meihua B, Ashcom J, Feng J, et al: Molecular cloning and purification of Ac-TMP, a developmentally regulated putative tissue inhibitor of metalloprotease released in relative abundance by adult Ancylostoma hookworm. Am J Trop Med Hyg 2002, 66:238-244.
26. Zhan B, Gupta R, Wong S P Y, Bier S, Jiang D, et al: Molecular cloning and characterization of Ac-TMP-2, a tissue inhibitor of metalloproteinase secreted by adult Ancylostoma caninum. Mol Biochem Parasitol 2008, 162:142-148.
27. Mulvenna J, Hamilton B, Nagaraj S H, Smyth D, Loukas A, et al: Proteomics analysis of the excretory/secretory component of the blood-feeding stage of the hookworm, Ancylostoma caninum. Mol Cell Proteomics 2009, 8:109-121.
28. Margulies M, Egholm M, Altman W E, Attiya S, Bader J S, et al: Genome sequencing in microfabricated high-density picolitre reactors. Nature 2005, 437:376-380.
29. Bentley D R, Balasubramanian S, Swerdlow H P, Smith G P, Milton J, et al: Accurate whole human genome sequencing using reversible terminator chemistry. Nature 2008, 456:53-59.
30. Harris T D, Buzby P R, Babcock H, Beer E, Bowers J, et al: Single-molecule DNA sequencing of a viral genome. Science 2008, 320:106-109.
31. Cantacessi C, Campbell B E, Gasser R B: Key strongylid nematodes of animals—impact of next-generation transcriptomics on systems biology and biotechnology. Biotechnol Adv 2012, 30:469-488.
32. Cantacessi C, Mitreva M, Jex A R, Young N D, Campbell B E, et al: Massively parallel sequencing and analysis of the Necator americanus transcriptome. PLoS Negl Trop Dis 2010, 4:e684.
33. Cantacessi C, Young N D, Nesjum P, Jex A R, Campbell B E, et al: The transcriptome of Trichuris suis—first molecular insights into a parasite with curative properties for key immune diseases of humans. PLoS One 2011, 6:e23590.
34. Jex A R, Liu S, Li B, Young N D, Hall R S, et al: Ascaris suum draft genome. Nature 2011, 479:529-533.
35. Mitreva M, Jasmer J P, Zarlenga D S, Wang Z, Abubucker S, et al: The draft genome of the parasitic nematode Trichinella spiralis. Nat Genet 2011, 43228-235.
37. Berriman M, Haas B J, LoVerde P T, Wilson R A, Dillon G P, et al: The genome of the blood fluke Schistosoma mansoni. Nature 2009, 460:352-358.
38. The Schistosoma japonicum Genome Sequencing and Functional Analysis Consortium: The Schistosoma japonicum genome reveals features of host-parasite interplay. Nature 2009, 460:345-351.
39. Young N D, Campbell B E, Hall R S, Jex A R, Cantacessi C, et al: Unlocking the transcriptomes of two carcinogenic parasites, Clonorchis sinensis and Opisthorchis viverrini. PLoS Negl Trop Dis 2010, 4:e719.
40. Young N D, Jex A R, Li B, Liu S, Yang L, et al: Whole genome sequence of Schistosoma haematobium. Nat Genet 2012, 44:221-225.
41. Wang X, Chen W, Huang Y, Sun J, Men J, et al: The draft genome of the carcinogenic human liver fluke Clonorchis sinensis. Genome Biol 2011, 12:R107.
42. Cantacessi C, Jex A R, Hall R S, Young N D, Campbell B E, et al: A practical, bioinformatic workflow system for large data sets generated by next-generation sequencing. Nucleic Acids Res 2010, 38:e171.
43. Young N D, Hall R S, Jex A R, Cantacessi C, Gasser R B: Elucidating the transcriptome of Fasciola hepatica—a key to fundamental and biotechnological discoveries for a neglected parasite. Biotechnol Adv 2010, 28:222-231.
44. Young N D, Jex A R, Cantacessi C, Hall R S, Campbell B E, et al: A portrait of the transcriptome of the neglected trematode, Fascia gigantica—biological and biotechnological implications. PLoS Negl Trop Dis 2011, 5:e1004.
45. Flicek P, Ridwan Amode M, Barrell D, Beal K, Brent S, et al: Ensembl 2012. Nucleic Acids Res 2012, 40:D84-D90.
46. Ghedin E, Wang S, Spiro D, Caler E, Zhao Q, et al: Draft genome of the filarial nematode parasite Brugia malayi. Science 2007, 317:1756-1760.
48. Altschul S F, Gish W, Miller W, Myers E W, Lipman D J: Basic Local Alignment Search Tool. J Mol Biol 1990, 215:403-410.
49. Hunter S, Jones P, Mitchell A, Apweiler R, Attwood T K, et al: InterPro in 2011: new developments in the family and domain prediction database. Nucleic Acids Res 2012, 40:D306-D312.
50. Docherty A J, Lyons A, Smith B J, Wright E M, Stephens P E, et al: Sequence of human tissue inhibitor of metalloproteinases and its identity to erythroid-potentiating activity. Nature 1985, 318:66-69.
51. Bendtsen J D, Nielsen H, von Heijne G, Brunak S: Improved prediction of signal peptides: SignalP 3.0. J Mol Biol 2004, 340:783-795.
52. Chen Y, Zhang Y, Yin Y, Gao G, Li S, et al: SPD—a web based secreted protein database. Nucleic Acids Res 2005, 33:D169-D173.
53. Choo K H, Tan T W, Ranganathan S: SPdb—a signal peptide database. BMC Bioinformatics 2005, 6:249.
54. Wang C K, Broder U, Weeratunga S K, Gasser R B, Loukas A, et al: SBAL: a practical tool to generate and edit structure-based amino acid sequence alignments. Bioinformatics 2012, 28:1026-1027.
55. McGuffin L J, Bryson K, Jones D T: The PSIPRED protein structure prediction server. Bioinformatics 2000, 16:404-405.
56. Ronquist F, Huelsenbeck J: MRBAYES 3: Bayesian phylogenetic inference under mixed models. Bioinformatics 2003, 19:1572-1574.
57. Tamura K, Peterson D, Peterson N, Stecher G, Nei M, et al: MEGA5: Molecular Evolutionary Genetics Analysis using maximum likelihood, evolutionary distances, and maximum parsimony methods. Mol Biol Evol 2011, 28:2731-2739.
58. Lobley A, Sadowski M I, Jones D T: pGenTHREADER and pDomTHREADER: new methods for improved protein fold recognition and superfamily discrimination. Bioinformatics 2009, 25:1761-1767.
59. Sali A, Blundell T: Comparative protein modeling by satisfaction of spatial restraints. J Mol Biol 1993, 234:779-815.
60. Laskowski R, MacArthur M, Moss D, Thornton J: PROCHECK: A program to check the stereochemical quality of protein structures. J Appl Cryst 1993, 26:283-291.
61. DeLano W: The PyMOL Molecular Graphics System. 2002. http://www.pymol.org/.
62. Li R, Yu C, Li Y, Lam T W, Yiu S M, et al: SOAP2: an improved ultrafast tool for short read alignment. Bioinformatics 2009, 25:1966-1967.
63. Mortazavi A, Williams B A, McCue K, Schaeffer L, Wold B: Mapping and quantifying mammalian transcriptomes by RNA-Seq. Nat Methods 2008, 6:S22-S32.
64. Kucera K, Harrison L M, Cappello M, Modis Y: Ancylostoma ceylanicum excretory-secretory protein 2 adopts a netrin-like fold and defines a novel family of nematode proteins. J Mol Biol 2011, 408:9-17.
65. Bungiro R D Jr, Cappello M: Detection of excretory/secretory coproantigens in experimental hookworm infection. Am J Trop Med Hyg 2005, 73:915-920.
66. Bungiro R D Jr, Solis C V, Harrison L M, Cappello M: Purification and molecular cloning of and immunization with Ancylostoma ceylanicum excretory-secretory protein 2, an immunoreactive protein produced by adult hookworms. Infect Immun 2004, 72:2203-2213.
67. Alper S, Laws R, Lackford B, Boyd W A, Dunlap P, et al: Identification of innate immunity genes and pathways using a comparative genomics approach. Proc Natl Acad Sci USA 2008, 105:7016-7021.
68. Ding C, Cicuttini F, Li J, Jones G: Targeting IL-6 in the treatment of inflammatory and autoimmune diseases. Expert Opin Investig Drugs 2009, 18:1457-1466.
69. Cuellar C, Wu W, Mendez S: The hookworm tissue inhibitor of metalloproteases (Ac-TMP-1) modifies dendritic cell function and induces generation of CD4 and CD8 suppressor T cells. PLoS Negl Trop Dis 2009, 3:e439.
70. Guyot R, Magre S, Leduque P, Le Magueresse-Battistoni B: Differential expression of tissue inhibitor of metalloproteinases type 1 (TIMP-1) during mouse gonad development. Dev Dyn 2003, 227:357-366.
71. Jaworski D M, Beem-Miller M, Lluri G, Barrantes-Reynolds R: Potential regulatory relationship between the nested gene DDC8 and its host gene tissue inhibitor of metalloproteinase-2. Physiol Genomics 2007, 28:168-178.
72. Sharpe R M: Regulation of spermatogenesis. In The physiology of reproduction. Edited by Knobil E, Neill J D. New York: Raven Press; 1994: 1363-1434.
73. Robinson L L, Sznajder N A, Riley S C, Anderson R A: Matrix metalloproteinases and tissue inhibitors of metalloproteinases in human fetal testis and ovary. Mol Hum Reprod 2001, 7:641-648.
74. Grima J, Calcagno K, Cheng C Y: Purification, cDNA cloning, and developmental changes in the steady-state mRNA level of rat testicular tissue inhibitor of metalloproteases-2 (TIMP-2). J Androl 1996, 17:263-275.
75. Fire A, Xu S, Montgomery M K, Kostas S A, Driver S E, et al: Potent and specific genetic interference by double-stranded RNA in Caenorhabditis elegans. Nature 1998, 391:806-811.
76. Islam M K, Miyoshi T, Yamada M, Tsuij N: Pyrophosphatase of the roundworm Ascaris suum plays an essential role in the worm's molting and development. Infect Immun 2005, 73:1995-2004.
77. Rinaldi G, Okatcha T I, Popratiloff A, Ayuk M A, Suttiprapa S, et al: Genetic manipulation of Schistosoma haematobium, the neglected schistosome. PLoS Negl Trop Dis 2011, 5:e1348.
78. Rinaldi G, Eckert S E, Tsai I J, Suttiprapa S, Kines K J, et al: Germline transgenesis and insertional mutagenesis in Schistosoma mansoni mediated by murine leukemia virus. PLoS Pathog 2012, 8:e1002810.
79. Brew K, Dinakarpandian D, Nagase H: Tissue inhibitors of metalloproteinases: evolution, structure and function. Biochim Biophys Acta, Prot Struct Mol Enzymol 2000, 1477:267-283.
80. Murphy G, Houbrechts A, Cockett M I, Williamson R A, O'Shea M, et al: The N-terminal domain of tissue inhibitor of metalloproteinases retains metalloproteinase inhibitory activity. Biochemistry 1991, 30:8097-8102.
81. Langton K P, Barker M D, McKie N: Localization of the functional domains of human tissue inhibitor of metalloproteinases-3 and the effects of a Sorsby's fundus dystrophy mutation. J Biol Chem 1998, 273:16778-16781.
82. De Clerck Y A, Yean T D, Lee Y, Tomich J M, Langley K E: Characterization of the functional domain of tissue inhibitor of metalloproteinase-2 (TIMP-2). Biochem J 1993, 289:65-69.
83. Willenbrock F, Crabbe T, Slocombe P M, Sutton C W, Docherty A J P, et al: The activity of the tissue inhibitors of metalloproteinases is regulated by C-terminal domain interactions: a kinetic analysis of the inhibition of gelatinase A. Biochemistry 1993, 32:4330-4337.
84. Ko Y C, Langley K E, Mendiaz E A, Parker V P, Taylor S M, et al: The C-terminal domain of tissue inhibitor of metalloproteinase-2 is required for cell binding but not for antimetalloproteinase activity. Biochem Biophys Res Commun 1997, 236:100-105.
85. Meng Q, Malinovskii V, Huang W, Hu Y J, Chung L, et al: Residue 2 of TIMP-1 is a major determinant of affinity and specificity for matrix metalloproteinases but effects of substitutions do not correlate with those of the corresponding P1′ residue of substrate. J Biol Chem 1999, 274:10184-10189.
86. Huang W, Suzuki K, Nagase H, Arumugam S, Van Doren S R, et al: Folding and characterisation of the amino-terminal domain of human tissue inhibitor of metalloproteinases-1 (TIMP-1) expressed at high yield in E. coli. FEBS Lett 1996, 384:155-161.
87. Amour A, Slocombe P M, Webster A, Butler M, Knight C G, et al: TNF-alpha converting enzyme (TACE) is inhibited by TIMP-3. FEBS Lett 1998, 435:39-44.
88. Kashiwagi M, Tortorella M, Nagase H, Brew K: TIMP-3 is a potent inhibitor of aggrecanase 1 (ADAM-TS4) and aggrecanase 2 (ADAM-TS5). J Biol Chem 2001, 276:12501-12504.
89. Olson M W, Gervasi D C, Mobahsery S, Fridman R: Kinetic analysis of the binding of human matrix metalloproteinase-2 and -9 to tissue inhibitor of matrix metalloproteinase (TIMP-1 and TIMP-2). J Biol Chem 1997, 272:29975-29983.
90. Hutton M, Willenbrock F, Brocklehurst K, Murphy G: Kinetic analysis of the mechanism of interaction of full-length TIMP-2 and gelatinase A—evidence for the existence of a low affinity intermediate. Biochemistry 1998, 37:10094-10098.
91. Lee M H, Atkinson S, Murphy G: Identification of the Extracellular Matrix (ECM) Binding Motifs of Tissue Inhibitor of Metalloproteinases (TIMP)-3 and Effective Transfer to TIMP-1. J Biol Chem 2007, 282:6887-6898.
92. Webster J P, Oliviera G, Rollinson D, Gower C M: Schistosome genomes: a wealth of information. Trends Parasitol 2010, 26:103-106.
93. Loukas A, Gaze S, Mulvenna J P, Gasser R B, Brindley P J, et al: Vaccinomics for the major blood feeding helminths of humans. OMICS 2011, 15:567-577.
94. Hagen J, Lee E F, Fairlie W D, Kalinna B H: Functional genomics approaches in parasitic helminths. Parasite Immunol 2012, 34:163-182.
95. Kumar S, Koutsovoulos G, Kaur G, Blaster M: Toward 959 nematode genomes. Worm 2012, 1:1-9.
96. Kalinna B H, Brindley P J: Manipulating the manipulators: advances in parasitic helminth transgenesis and RNAi. Trends Parasitol 2007, 23:197-204.
97. Selkirk M E, Huang S C, Knox D P, Britton C: The development of RNA interference (RNA) in gastrointestinal nematodes. Parasitology 2012, 139:605-612.

1. A method of reducing or alleviating inflammation in a subject, the method including the step of administering to the subject a therapeutically effective amount of an isolated protein comprising an amino acid sequence set forth in SEQ ID NO:20, or a biologically active fragment or variant thereof or combinations of these, to thereby reduce or alleviate inflammation in the subject.

2. The method of claim 1, wherein the inflammation is associated with, or secondary to, a disease, disorder and/or condition in the subject.

3. The method of claim 2, wherein the disease, disorder and/or condition is refractory to a baseline therapy.

4. The method of claim 3, wherein the baseline therapy comprises administration of at least one baseline agent selected from the group consisting of nonsteroidal anti-inflammatory drugs (NSAIDs), aminosalicylates, corticosteroids, immunosuppressants, anti-cytokine/cytokine receptor agents, antibiotics, and combinations thereof.

5. The method of claim 3, wherein, at least initially, the isolated protein or the biologically active fragment or variant thereof or combinations of these is administered adjunctively with the baseline therapy.

6. The method of claim 3, wherein, at least initially, the isolated protein or the biologically active fragment or variant thereof or combinations of these, is administered adjunctively with the at least one baseline agent, which is administered at less than a full dose.

7. A method for preventing inflammation in a subject, the method including the step of administering to the subject a therapeutically effective amount of an isolated protein comprising an amino acid sequence set forth in SEQ ID NO:20, or the biologically active fragment or variant thereof or combinations of these, to thereby prevent the inflammation in the subject.

8. The method of claim 7, wherein the inflammation is associated with, or secondary to a disease, disorder and/or condition in the subject.

9. The method of claim 8, wherein the disease, disorder and/or condition is an immunological disease, disorder and/or condition.

10. The method of claim 9, wherein the immunological disease, disorder and/or condition is selected from the group consisting of Addison's disease, ankylosing spondylitis, celiac disease, chronic inflammatory demyelinating polyneuropathy (CIDP), chronic recurrent multifocal ostomyelitis (CRMO), Crohn's disease, demyelinating neuropathies, glomerulonephritis, Goodpasture's syndrome, Graves' disease, Guillain-Barre syndrome, Hashimoto's encephalitis, Hashimoto's thyroiditis, hypogammaglobulinemia, idiopathic thrombocytopenic purpura (ITP), insulin-dependent diabetes (type 1), juvenile arthritis, Kawasaki syndrome, multiple sclerosis, myasthenia gravis, postmyocardial infarction syndrome, primary biliary cirrhosis, psoriasis, idiopathic pulmonary fibrosis, Reiter's syndrome, rheumatoid arthritis, sarcoidosis, scleroderma, Sjogren's syndrome, systemic lupus erythematosus (SLE), thrombocytopenic purpura (TTP), ulcerative colitis, vasculitis, vitiligo, and Wegener's granulomatosis.

11. The method of claim 8, wherein the disease is a disease of the digestive tract.

12. The method of claim 11, wherein the disease is chronic gastritis or an inflammatory bowel disease.

13. The method of claim 12, wherein the inflammatory bowel disease is Crohn's disease or ulcerative colitis.

14. The method of claim 8, wherein the disease is a disease of the respiratory system.

15. The method of claim 14, wherein the disease is selected from the group consisting of asthma, emphysema, chronic bronchitis, and chronic obstructive pulmonary disease (COPD).

16. The method of claim 1, further including the step of administering to the subject at least one additional agent.

17. The method of claim 16, wherein the at least one additional agent is selected from the group consisting of nonsteroidal anti-inflammatory drugs (NSAIDs), aminosalicylates, corticosteroids, immunosuppressants, anti-cytokine/cytokine receptor agents, antibiotics, and combinations thereof.

18. The method of claim 1, wherein the subject is a mammal.

19. The method of claim 18, wherein the mammal is a human.

20. A pharmaceutical composition comprising a therapeutically effective amount of an isolated protein comprising an amino acid sequence set forth in SEQ ID NO:20, or a biologically active fragment or variant thereof or combinations of these, together with a pharmaceutically acceptable carrier, diluent or excipient.

21. The pharmaceutical composition of claim 20 which further comprises at least one additional agent.

22. The pharmaceutical composition of claim 21, wherein the at least one additional agent is selected from the group consisting of nonsteroidal anti-inflammatory drugs (NSAIDs), aminosalicylates, corticosteroids, immunosuppressants, anti-cytokine/cytokine receptor agents, antibiotics, and combinations thereof.

23. The method of claim 2, wherein the disease, disorder and/or condition is an immunological disease, disorder and/or condition.

24. The method of claim 23, wherein the immunological disease, disorder and/or condition is selected from the group consisting of Addison's disease, ankylosing spondylitis, celiac disease, chronic inflammatory demyelinating polyneuropathy (CIDP), chronic recurrent multifocal ostomyelitis (CRMO), Crohn's disease, demyelinating neuropathies, glomerulonephritis, Goodpasture's syndrome, Graves' disease, Guillain-Barre syndrome, Hashimoto's encephalitis, Hashimoto's thyroiditis, hypogammaglobulinemia, idiopathic thrombocytopenic purpura (ITP), insulin-dependent diabetes (type1), juvenile arthritis, Kawasaki syndrome, multiple sclerosis, myasthenia gravis, postmyocardial infarction syndrome, primary biliary cirrhosis, psoriasis, idiopathic pulmonary fibrosis, Reiter's syndrome, rheumatoid arthritis, sarcoidosis, scleroderma, Sjogren's syndrome, systemic lupus erythematosus (SLE), thrombocytopenic purpura (TTP), ulcerative colitis, vasculitis, vitiligo, and Wegener's granulomatosis.

25. The method of claim 2, wherein the disease is a disease of the digestive tract.

26. The method of claim 25, wherein the disease is chronic gastritis or an inflammatory bowel disease.

27. The method of claim 26, wherein the inflammatory bowel disease is Crohn's disease or ulcerative colitis.

28. The method of claim 2, wherein the disease is a disease of the respiratory system.

29. The method of claim 28, wherein the disease is selected from the group consisting of asthma, emphysema, chronic bronchitis, and chronic obstructive pulmonary disease (COPD).


SEQUENCE LISTING (39 sequences)

SEQ ID NO: 1 - protein, 207 aa, Homo sapiens
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

SEQ ID NO: 2 - protein, 220 aa, Homo sapiens
MGAAARTLRLALGLLLLATLLRPADACSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDP

SEQ ID NO: 3 - protein, 211 aa, Homo sapiens
MTPWLGLIVLLGSWSLGDWGAEACTCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCNCKIKSCYYLPCFVTSKNECLWTDMLSNFGYPGYQSKHYACIRQKGGYCSWYRGWAPPDKSIINATDP

SEQ ID NO: 4 - protein, 224 aa, Homo sapiens
MPGSPRPAPSWVLLLRLLALLRPPGLGEACSCAPAHPQQHICHSALVIRAKISSEKVVPASADPADTEKMLRYEIKQIKMFKGFEKVKDVQYIYTPFDSSLCGVKLEANSQKQYLLTGQVLSDGKVFIHLCNYIEPWEDLSLVQRESLNHHYHLNCGCQITTCYTVPCTISAPNECLWTDWLLERKLYGYQAQHYVCMKHVDGTCSWYRGHLPLRKEFVDIVQP

SEQ ID NO: 5 - protein, 220 aa, Gallus gallus
MPGAALPSLLAWLAVLLLGRARPADACSCSPIHPQQAFCNADVVIRAKRVSAKEVDSGNDIYGNPIKRIQYEVKQIKMFKGPDQDIEFIYTAPSTEVCGQPLDTGGKKEYLIAGKSEGDGKMHITLCDLVATWDSVSPTQKKSLNQRYQMGCECKISRCLSIPCFVSSSDECLWTDWAMEKIVGGRQAKHYACIKRSDGSCAWYRGMAPPKQEFLDIEDP

SEQ ID NO: 6 - protein, 220 aa, Canis familiaris
MGAAARSLRLALGLLLLATLPRPADACSCSPVHPQQAFCNADVVIRAKAVSVKEVDSGNDIYGNPIKRIQYEIKQIKMFKGPDKDIEFIYTAPSSAVCGVSLDIGGKKEYLIAGKAEGNGKMHITLCDFIVPWDTLSSTQKKSLNHRYQMGCECKITRCPMIPCYISSPDECLWMDWVTEKSINGHQAKFFACIKRSDGSCAWYRGAAPPKQEFLDIEDP

SEQ ID NO: 7 - protein, 194 aa, Oryctolagus cuniculus
CSCSPVHPQQAFCNADIVIRAKAVNKKEVDSGNDIYGNPIKRIQYEIKQIKMFKGPDQDIEFIYTAPSSAVCGVSLDIGGKKEYLIAGKAEGNGNMHITLCDFIVPWDTLSATQKKSLNHRYQMGCECKITRCPMIPCYISSPDECLWMDWVTEKNINRHQAKFFACIKRSDGSCAWYRGAAPPKQEFLDIEDP

SEQ ID NO: 8 - protein, 210 aa, Drosophila melanogaster
MDLRKHLGLLTLLLVAVFAFYGRPADACSCMPSHPQTHFAQADYVVQLRVLRKSDTIEPGRTTYKVHIKRTYKATSEARRMLRDGRLSTPQDDAMCGINLDLGKVYIVAGRMPTLNICSYYKEYTRMTITERHGFSGGYAKATNCTVTPCFGERCFKGRNYADTCKWSPFGKCETNYSACMPHKVQTVNGVISRCRWRRTQLYRKCMSNP

SEQ ID NO: 9 - protein, 210 aa, Drosophila melanogaster; identical to SEQ ID NO: 8

SEQ ID NO: 10 - protein, 210 aa, Drosophila melanogaster; identical to SEQ ID NO: 8

SEQ ID NO: 11 - protein, 210 aa, Drosophila melanogaster; identical to SEQ ID NO: 8

SEQ ID NO: 12 - protein, 210 aa, Drosophila melanogaster; identical to SEQ ID NO: 8

SEQ ID NO: 13 - protein, 205 aa, Mus musculus
MMAPFASLASGILLLLSLIASSKACSCAPPHPQTAFCNSDLVIRAKFMGSPEINETTLYQRYKIKMTKMLKGFKAVGNAADIRYAYTPVMESLCGYAHKSQNRSEEFLITGRLRNGNLHISACSFLVPWRTLSPAQQRAFSKTYSAGCGVCTVFPCLSIPCKLESDTHCLWTDQVLVGSEDYQSRHFACLPRNPGLCTWRSLGAR

SEQ ID NO: 14 - protein, 205 aa, Mus musculus; identical to SEQ ID NO: 13

SEQ ID NO: 15 - protein, 211 aa, Mus musculus
MTPWLGLVVLLSCWSLGHWGAEACTCSPSHPQDAFCNSDIVIRAKVVGKKLVKEGPFGTLVYTIKQMKMYRGFSKMPHVQYIHTEASESLCGLKLEVNKYQYLLTGRVYEGKMYTGLCNFVERWDHLTLSQRKGLNYRYHLGCNCKIKSCYYLPCFVTSKNECLWTDMLSNFGYPGYQSKHYACIRQKGGYCSWYRGWAPPDKSISNATDP

SEQ ID NO: 16 - protein, 224 aa, Mus musculus
MPWSPLAALSWALVLRLLALLWPPGRGEACSCAPAHPQQHFCHSALVIRAKISSEKVVPASKDPADTQKLIRYEIKQIKMFKGFEKAKDIQYVYTPFDSSLCGVKLETNSHKQYLLTGQILSDGKVFIHLCNYIEPWEDLSLVQRESLNHHYHQNCGCQITTCYAVPCTISAPNECLWTDWLLERKLYGYQAQHYVCMKHVDGICSWYRGHLHLRKEYVDIIQP

SEQ ID NO: 17 - protein, 220 aa, Mus musculus
MGAAARSLRLALGLLLLATLLRPADACSCSPVHPQQAFCNADVVIRAKAVSEKEVDSGNDIYGNPIKRIQYEIKQIKMFKGPDKDIEFIYTAPSSAVCGVSLDVGGKKEYLIAGKAEGDGKMHITLCDFIVPWDTLSITQKKSLNHRYQMGCECKITRCPMIPCYISSPDECLWMDWVTEKSINGHQAKFFACIKRSDGSCAWYRGAAPPKQEFLDIEDP

SEQ ID NO: 18 - protein, 158 aa, Caenorhabditis elegans
MQNLSLSLVILSVLIAVTLACKCREQSTKESFCNAHWVSHVKVKVRVGKQGLPEGSERKGLNNLRYTVQHVEVFKKPSNMTTLPDEIFTPSEAPACGLKIAAGHEYLLAGRVEGPNALYTVLCGQVLPDDRSQTSFENVLEWKNVPQTLQSQVKSIKC

SEQ ID NO: 19 - protein, 132 aa, Ancylostoma duodenale
MRTAIVLLAFFATASTNLYCPNADKITRKDVKSSEFVALVKVESSVAKGDRYNEYFEYGVKYEKLFQWWHYWVAEATIIRDAPKTLKVSQNCGVRLELGKGYVLGCRTFSHCHFVRPYNNLTTEEMELIQKQ

SEQ ID NO: 20 - protein, 137 aa, Necator americanus
MKYFVIVAACVTVSNACSCLPFGTPKESFCSSDFVSHVKVISKKDPNTSPDGLQDITYVVQHICVYRKPSKIKKLSNQIVTASNSAACGIELDVGVEYLLGGSVDEKGVVRSFLCGIVEKWSDVPSKEKADLGKYKC

SEQ ID NO: 21 - protein, 139 aa, Necator americanus
MSKILILIVACVATSEACSCLPFPGLQEAFCASDFVSRVKVISKKDPNAYSKGFQDVIYTVHHIHVYRKPSKIAKLPSEIYTAPNSAACGIELEIGKEYLIGGSVDEQGKLHSYLCGILQKWHEVPKKDRVALRTYKCY

SEQ ID NO: 22 - protein, 112 aa, Necator americanus
MIHVFILCFTLLLYFPYTDGCFCSPAQSTKESFCKADWVSHAKILGTSNSGRRNDLNGISYMVEHKDVFKMPTKVKMLPVEINTALADTACGVKLKIGAEYLLAGGWFGLLF

SEQ ID NO: 23 - protein, 79 aa, Oesophagostomum dentatum
  misc_feature (1)..(1): Xaa can be any naturally occurring amino acid
XPLDSHPICSLTLVCLKILCGFXIPPGLSSSSEEIFTPKHSATCGLQLDVGKEYLLAGKSNDGVLRVISCGQIISDDPX

SEQ ID NO: 24 - protein, 80 aa, Oesophagostomum dentatum
  misc_feature (1)..(1): Xaa can be any naturally occurring amino acid
XLIFCLCIIWTEACFCTPAQSTKESFCKADWVSHVKVLSTTSSTRNINDVTYLLWHKEVFKSPLSIKQLPVRVHAVLLES

SEQ ID NO: 25 - protein, 175 aa, Dictyocaulus filaria
  misc_feature (3)..(3): Xaa can be any naturally occurring amino acid
QVXEMNVLLIVSFTLCISLTSSCQCETQTSQESFCAADWVSHVKVKFRISKQPLPVGLSRRGLNNIRYSVNHIDIYKKPSNMTEMPTDIYTPSESPACGLTIIGAGKEYLLAGRVLNGTLYTVLCGQILPDNPSKESYELVLEWQRVPKPLLEKLNKFTCXSLIKSMFCFLFHFX

SEQ ID NO: 26 - protein, 175 aa, Ascaris suum
MQITKLQLVFIVIECLFFAATYGCTCQSQTPLQSYCRADWVSHLRVKFRVTKQPMPNNPLRKGFTNIRYGAHHIRVYKVPPQFANSTLPTEIFTPSEAAACGLVLEAGKEYLLAGRVRNGTLTTALCGQIAGNHPGTSSFESILEWTMVSERLQQWLEKDNSALCPEILGAQVPS

SEQ ID NO: 27 - protein, 152 aa, Ascaris suum
MKHHLTQFLLFSLLIVALKACKCGNLSPKEAYSNAQWVTRAKILRRQSKKIDGIGYIEYKVNHIDVFKNIRKPRKLPDEITTLATSASCGLPSLGIGKEYLLSGRFVDGDILRMYACEQIIVKIKDGKPLGVVLEWNEVPNDLRKKLSGGNV

SEQ ID NO: 28 - protein, 150 aa, Ascaris suum
MQQLARFLVCCLLIVAVDACKCRVLTAQEAFKNAEWVSRVKILRKEEKTEGGVDHIEYEVEHVEIFKNSRSPQLSKEITTSTSTAACGLVSLQIGKEYLLSGRFTDRDYLHINSCGQITDSAKNGQPLGFVLEWNEVPNDLKGKLSDGSV

SEQ ID NO: 29 - protein, 359 aa, Schistosoma haematobium
MEASPSYYLLFIILFIITLYIEQTKEDIKVHTAVQIKRPSNRHWIERASSARQITTPSVLTTTITSNRFIYSGNVSSINMSSEVIKNSVRRKELQDQPQLPNIYSQTYLDYQGNRERPIDEYNQPNILYPHEGLNAFHNLPSWYSLRYRRYPREEPNHPYDDNNLQLPWNYKPKCRSCGRPLLQDSFCIDDFVLRVYIYKESVEPNGQRHYLAYIYQVYKDIKHYMQPTLVRQIVYNCDRFQTGPMLGEVYLITGIYISGVPHVDSCSWKAPWNQVTREQKRYLRNRYSIQCGICQLQRVLFVDPIYHRNPKTCYLPISPNSNEMMCRDKFSLCRLQRRTNRCNFINNKSYRICETWST

SEQ ID NO: 30 - protein, 202 aa, Schistosoma mansoni
MEASPPYYLLFIILLIITLYIEQTKEDIKVHTSVQIRRPSNRHWIERASSARQKTTPSVLTTTIIPNRFIYLGNASSINMSSELKKNSIRRKELQDQPQLPNTNSPTYLDYQGSRKHPIDEYNQHSILYPHEGLSAFHNLPSWYSLRYRRYPREEPNHPYDDNNLQLPWNYKPKCRSCGRPLLQDSFCIDDFANFDGAMKIK

SEQ ID NO: 31 - protein, 190 aa, Schistosoma japonicum
MEILSFILFYTLVYIDQTKEDIKVHTALQVKHPSNQHWIERTSSARYVTPPTVRTTSYRLHNAENSLSINLSSDLKKNTFRRKELQDQPQLPNVYPQAYLNYPINRKLPNDEYNQHEIIYPYGVMNPLHNLPSWYSLRYRRYPREEPHHPYNDNNLQLPWNYKPKCRSCGRPLLQDSFCVDDFGKHLFVV

SEQ ID NO: 32 - protein, 140 aa, Ancylostoma caninum
MRVAIVFIACFAVAHACKCEKKPRPPLEKLLCQSQFVTHAKVTKKRIDGYFIYYDLEHKEVYKPKDRSIPIELFSWREKENCGVPDLEEGKEYLIGGKVTDYGDGDLVISVSRCDLLRNWTDVSGEEKKLLGTFKCENQS

SEQ ID NO: 33 - protein, 244 aa, Ancylostoma caninum
MISLIVFIACLTTTQAACSCKPFGTLKEAFCQSDYVLLAKVLSVNSKYGESSRNEANDMSTTANGTWSYHVWHMRTWKGPVVDTSVLTTSYSECGVTGLLKNWDYFLTGKQGKDGEITITSCDFVMPSTDVTPEEHDLLMDLMGDPKKCEEKDDERDVKENENSVEENDEKDEEENGEKTVEENDEKTVEENDEKVEEENGEKTVEENDEKTVEENDEKDEEENGEKTVEENDEKTVEENDEQE

SEQ ID NO: 34 - protein, 114 aa, Necator americanus
MRGLLILVICLSSAYRSSCKSSGVCRYYGFAVQAKVTRVSSACCTPAYPQGRFIYKVEIEETYKGDMEYGKSYELYTSPSSCECTGLDRDETYLLIGYADDGSRKLSLCVSKYA

SEQ ID NO: 35 - protein, 118 aa, Necator americanus
MNIGLVSHVKVLYKMILPNNGLDDVLYIIQHIQTYKSSSGVHNMYSYVLTSSESSACGLVLDVGKEYLLSGTFYNGEYHTSTCFQVVTDDPADNFSGNLMEWKDVTSAFENRLHSFQC

SEQ ID NO: 36 - protein, 114 aa, Necator americanus
MTKQPLPAGSSRKGLNNIRYAVNHERIYKRPPGVSNLPAEIYTPSEPPACGLIIDAGKEYLLAGQMHNGTLHTVLCGQVPVDNPTEALYENVLEWKNVPKELQEKLPFDCAKNE

SEQ ID NO: 37 - protein, 106 aa, Necator americanus
MDFVSHVKVLEQNSDNSKMVSLLVYSVEHLDVFKKPHSDELPREIKTPSLSGFCGARLKVGEEYLLGGMIDNDGVANISLCGLNKEWNALEPNEKEELATYRCERR

SEQ ID NO: 38 - protein, 86 aa, Necator americanus
MIATIHTIWADVVGKTALGGGDKGYYKYEVVSKEIYKGEMMRGKSYDLSMSYPNSSCAGLTVGKTYLLAGTSDDNDRKQISLCRIL

SEQ ID NO: 39 - protein, 106 aa, Ancylostoma ceylanicum
GSHMEYCPKMLSEIRQEDINDVETVAYVTVTGKTARSYNLQYWRLYDVPKTAPSQWPSFGTLRDDCGNIQLTADTDYVLGCKSGNQDCFVKLHDGLSQKEKDLLKE